(19) United States
(12) Patent Application Publication
(10) Pub. No.: US 2005/0277199 A1
(43) Pub. Date: Dec. 15, 2005

(54) Single-pass compound purification and analysis

(75) Inventors: John Isbell, San Diego, CA (US); Matthew Rynd, Carlsbad, CA (US); Tessa Rynd, Carlsbad, CA (US); Ding Yuan, San Diego, CA (US); James Kevin Mainquist, San Diego, CA (US); Andrew Joseph Meyer, Solana Beach, CA (US)

Correspondence Address: QUINE INTELLECTUAL PROPERTY LAW GROUP, P.C., P O BOX 458, ALAMEDA, CA, 94501, US

(73) Assignee: IRM LLC, Hamilton (BM)

(21) Appl. No.: 11/137,863

(22) Filed: May 24, 2005

Related U.S. Application Data
(60) Provisional application No. 60/574,705, filed on May 25, 2004.

(51) Int. Cl. G06F 19/00; G01N 31/00; C12P 21/06; G01N 30/02

(52) U.S. Cl. 436/161; 702/22

(57) ABSTRACT

The present invention relates to high-throughput compound purification and analysis systems that include representative sample fraction storage components that are structured to at least transiently store representative sample fraction aliquots prior to analysis. In addition, related computer program products and methods are also provided.

### CROSS-REFERENCES TO RELATED APPLICATIONS

This application claims the benefit of U.S. Provisional Application No. 60/574,705, filed May 25, 2004, the disclosure of which is incorporated by reference in its entirety for all purposes.

### FIELD OF THE INVENTION

The present invention relates generally to compound purification and analysis. In addition to purification and analytical systems, related computer program products and methods are also provided.

### BACKGROUND OF THE INVENTION

Chromatographic separations that provide purified compounds for evaluation are an important aspect of many combinatorial synthesis platforms. Oftentimes, compounds to be purified are presented to the purification system in the wells of standard multi-well containers in an effort to enhance throughput. In addition, preparatory scale purification is typically employed with some form of detection (e.g., mass spectroscopic detection, ultraviolet/visible wavelength (UV/Vis) detection, luminescence detection, evaporative light-scattering (ELS) detection, refractive index (RI) detection, electrochemical detection, chemiluminescence nitrogen (CLN) detection, and/or the like) to effect the collection of fractions that contain the compounds of interest.

After a preparative purification process (e.g., an LC/MS process) is performed, it is often desirable to perform post-purification analysis on collected sample fractions to determine the relative purities of the fractions. Traditionally, this is done after a series of samples has been purified and it may be done with the same or a different LC(/MS) system. In these processes, collected fractions are generally inhomogeneous upon collection in the absence of sufficient agitation or if insufficient time is provided for the fractions to reach homogeneity. This lack of homogeneity can be a source of inaccurate purity data being obtained for these collected sample fractions.

Traditional approaches to post-purification analysis of collected sample fractions typically significantly limit the throughput of the overall process. For example, many conventional methodologies include evaporating the collected sample fractions and redissolving them in a set volume or to a theoretical concentration prior to obtaining any information about the purity of the collected sample fractions. This tends to be a time consuming and labor intensive process, particularly when a preparative purification scheme has been performed on a large library of compounds.

### SUMMARY OF THE INVENTION

The present invention relates generally to compound purification and analysis. In certain embodiments, for example, the systems and methods described herein provide the ability to not only sample a proportional or representative amount from each fraction as it is collected, but also to analyze the representative aliquot immediately after each preparative or semi-preparative chromatographic run is completed to provide post-purification quality control data within the same purification run. The flow stream sampling and analysis components of the systems described herein reliably sample amounts that are proportional to the amounts of material collected during fraction collection events. This permits immediate analysis of a proportionally sampled amount (i.e., a representative sample fraction aliquot) from the flow stream, thereby removing additional agitation or evaporation and reconstitution steps associated with pre-existing approaches. Accordingly, the throughput of purification and analysis processes performed using the systems described herein is typically considerably improved relative to these convention techniques.

In one aspect, the invention provides a purification system that includes at least a first chromatographic component (e.g., at least one chromatography column, etc.) configured to separate sample components from one another and at least one fraction collection component that is structured to collect at least one sample fraction. The first chromatographic component is a serial device in certain embodiments. In some embodiments, the purification system includes at least one handling component that is configured to convey samples into an inlet of the first chromatographic component. The system also includes at least one representative sample fraction storage component that is structured to at least transiently store at least one representative sample fraction aliquot. In addition, the purification system also includes at least one splitting mechanism in fluid communication with the first chromatographic component, the fraction collection component, and the representative sample fraction storage component. The splitting mechanism is structured to split sample fractions flowed toward the fraction collection component such that substantially representative aliquots of the sample fractions are flowed to the representative sample fraction storage component. Typically, the purification system includes at least one pump operably connected to at least one system component, which pump is configured to convey fluidic materials in the system.

In some embodiments, the fraction collection component comprises at least one holder including a base, a coupling mechanism, and a top plate including a plurality of apertures in which the coupling mechanism couples the base to the top plate in at least one position. Typically, the top plate and the base have disposed between them one or more structures collectively comprising a plurality of external processing regions. In certain embodiments, at least one body structure is disposed on the top plate such that the top plate is between the body structure and the structures comprising the external processing regions. The body structure includes a plurality of first access apertures connected to, and separated from, a plurality of second access apertures by a plurality of inner cavities. The inner cavities include a plurality of internal processing regions. In addition, the body and the structures are removably sealed such that the internal processing regions are removably sealed to the external processing regions.

The representative sample fraction storage components of the purification systems include various embodiments. To illustrate, the splitting mechanism typically fluidly communicates with the fraction collection component and the representative sample fraction storage component via at least one conduit. In these embodiments, at least a portion of the conduit optionally comprises the representative sample fraction storage component. To further illustrate, the purification system includes one or more switching valves in fluid communication with at least one system component. In these embodiments, the representative sample fraction storage component optionally comprises at least one fluid loop disposed at least partially in at least one of the switching valves.

The purification systems each generally include at least one controller operably connected to at least one system component, which controller controls operation of the system component. In some embodiments, for example, the controller comprises at least one computer having one or more logic instructions that effect one or more of: receiving one or more user input parameters selected from the group consisting of: sample input volumes, fluid flow rates, solvent selection, gradient selection, flow path selection, representative sample fraction aliquot volumes, collected sample fraction volumes, delay time between sample component detection and sample fraction aliquot collection and/or storage, at least one fraction collection component volume capacity, and output displays; tracking samples, sample fractions, and/or representative sample fraction aliquots; receiving and storing data from the system component; correlating data received from the system component with one or more sample fractions collected by the fraction collection component; processing data to produce processed data; terminating further processing of one or more sample fractions and/or another sample (e.g., the next sample in a purification queue) if processed data fails to meet one or more selected criteria; quantifying one or more sample fraction components; altering a duration of time between detection of separated sample components and triggering sample fraction collection and representative sample fraction aliquot storage; altering a delay duration between detection of separated sample components and triggering sample fraction collection; altering a duration of time between triggering sample fraction collection and triggering representative sample fraction aliquot storage; or, conveying samples into an inlet of the first chromatographic component.

In some embodiments, the purification system includes at least a second chromatographic component (e.g., at least one chromatography column, etc.) in fluid communication with the representative sample fraction storage component. The second chromatographic component is configured to separate components of representative sample fraction aliquots from one another when the representative sample fraction aliquots are conveyed from the representative sample fraction storage component into an inlet of the second chromatographic component. The second chromatographic component and the representative sample fraction storage component generally together comprise a parallel device (i.e., they are typically capable of operating substantially simultaneously with one another). The first and second chromatographic components comprise identical or different types of chromatographic components. In certain embodiments, for example, the first and/or second chromatographic component comprises a supercritical fluid chromatographic component. In some embodiments, the system includes at least one detection component that communicates with both the first and second chromatographic components. Exemplary detection components include a mass spectrometer (MS), a UV/Vis detector, an evaporative light-scattering detector (ELSD), a nuclear magnetic resonance (NMR) detector, an electrochemical detector, a fluorescence detector, a chemiluminescent nitrogen detector, a refractive index detector, a thermal conductivity detector, a flame ionization detector, a photoionization detector, an electron capture detector, a radiation detector, a weight scale, and/or the like.

In another aspect, the invention provides a computer program product that includes a computer readable medium having one or more logic instructions that effect collection of at least a first aliquot of at least one sample fraction in at least one fraction collection component of a purification system and at least transient storage of at least a second aliquot of the sample fraction in at least one representative sample fraction storage component of the purification system when the sample fraction is split into at least two aliquots that are substantially representative of one another in the purification system. Typically, the computer program product includes at least one logic instruction that effects separation of two or more components of a sample from one another with at least one chromatographic component of the purification system to produce the sample fraction. In some embodiments, the computer program product includes at least one logic instruction that effects one or more of: receiving one or more user input parameters selected from the group consisting of: sample input volumes, fluid flow rates, solvent selection, gradient selection, flow path selection, delay time between sample component detection and sample fraction aliquot collection and/or storage, at least one fraction collection component volume capacity, and output displays; tracking samples, sample fractions, and/or representative sample fraction aliquots; receiving and storing data from at least one component of the purification system; altering a duration of time between detection of separated sample components and triggering sample fraction collection and representative sample fraction aliquot storage; altering a delay duration between detection of separated sample components and triggering sample fraction collection; altering a duration of time between triggering sample fraction collection and triggering representative sample fraction aliquot storage; or correlating the data received from the purification system component with one or more sample fractions collected by the fraction collection component. To illustrate, the computer readable medium optionally comprises one or more of: a CD-ROM, a floppy disk, a tape, a flash memory device or component, a system memory device or component, a hard drive, a data signal embodied in a carrier wave, or the like.

In certain embodiments, the computer program product includes at least one logic instruction that effects detection of at least one property of the sample fraction, the first aliquot of the sample fraction, and/or the second aliquot of the sample fraction with at least one detection component of the purification system. In some of these embodiments, the computer program product includes at least one logic instruction for correlating the detected property of the second aliquot of the sample fraction with the first aliquot of the first sample fraction collected in the fraction collection component to produce correlation data. Optionally, the computer program product includes at least one logic instruction that effects storage of the correlation data for the first and second aliquots in an identical data file in at least one data storage medium. In some embodiments, the computer program product includes at least one logic instruction for automatically processing the correlation data to produce processed correlation data. In these embodiments, the computer program product optionally includes at least one logic instruction that effects termination of further processing (e.g., purifying, agitating, weighing, evaporating, lyophilizing, storing, screening, etc.) of the sample fraction and/or another sample if the processed correlation data fails to meet one or more selected criteria, such as a level of purity. In these embodiments, the computer program product optionally also includes at least one logic instruction that effects introduction of another sample into the purification system for processing.

In some embodiments, the computer program product includes at least one logic instruction that effects separation of components of the second aliquot of the sample fraction from one another with at least one chromatographic component of the purification system. In these embodiments, the computer program product optionally includes at least one logic instruction that effects detection of the separated components of the second aliquot of the sample fraction with at least one detection component of the purification system.

To further illustrate, the computer program product includes at least one logic instruction that effects quantification of at least one component of the sample fraction. In some of these embodiments, for example, the computer program product includes at least one logic instruction for altering a duration of first aliquot collection to alter a quantity of the component collected in the fraction collection component.

In still another aspect, the invention relates to a method of storing a sample fraction aliquot. The method includes (a) separating two or more components of the sample from one another to produce at least first and second sample fractions, and (b) splitting at least the first sample fraction into at least two aliquots, which aliquots are substantially representative of one another. Typically, (a) is performed using at least one chromatographic component (e.g., at least one chromatography column, etc.). The method also includes (c) collecting at least a first aliquot of the first sample fraction in at least one fraction collection component, and (d) storing at least a second aliquot of the first sample fraction at least transiently in at least one representative sample fraction storage component, thereby storing the sample fraction aliquot. Generally, (c) and (d) are performed substantially simultaneous with one another. In some embodiments, the method includes optimizing an amount of the first sample fraction that is collected in the fraction collection component. In certain embodiments, the method includes quantifying at least one component of the first and/or second sample fractions. Optionally, the method includes altering a duration of (c) to alter a quantity of the first sample fraction collected in the fraction collection component. In some embodiments, for example, (c) includes detecting the first sample fraction and triggering collection of the first sample fraction in the fraction collection component. The duration of (c) is optionally altered by altering a delay duration between detection of the first sample fraction and triggering the collection of the first sample fraction. Typically, the second aliquot of the first sample fraction comprises less than 50% (e.g., about 40%, about 20%, about 10%, about 5%, or less) of a total volume of the first sample fraction. In some embodiments, the method includes further processing the first aliquot of the first sample fraction collected in the fraction collection component. In certain embodiments, the method includes tracking the sample, the sample fractions, and/or aliquots of the sample fractions.

In some embodiments, the method of processing a sample includes (e) detecting at least one property of the first sample fraction, the second sample fraction, the first aliquot of the first sample fraction, and/or the second aliquot of the first sample fraction. In certain embodiments, for example, the method includes detecting the property of the first sample fraction, the second sample fraction, the first aliquot of the first sample fraction, and/or the second aliquot of the first sample fraction using at least one identical detection component (e.g., the same set of UV/Vis, ELSD, and MS detectors). Typically, the method includes performing (e) prior to separating components of another sample. In some embodiments, (e) includes (i) separating components of the second aliquot of the first sample fraction from one another, and (ii) detecting the separated components. In these embodiments, the method generally includes performing (i) using at least one chromatographic component, such as a chromatography column or the like). In other embodiments, sample components are separated from one another using other separation techniques and devices (e.g., capillary electrophoresis devices, etc.).

To further illustrate, the method includes correlating the detected property of the second aliquot of the first sample fraction with the first aliquot of the first sample fraction collected in the fraction collection component to produce correlation data in some embodiments. This generally eliminates the need to separately detect properties, such as a purity level of the first aliquot of the first sample fraction collected in the fraction collection component. In certain embodiments, the method includes automatically processing the correlation data to produce processed correlation data. In these embodiments, the method optionally includes terminating further processing of at least the first sample fraction and/or another sample if the processed correlation data fails to meet one or more selected criteria (e.g., a level of purity, etc.). In some embodiments, the method includes storing the correlation data in at least one data storage medium (e.g., a database, etc.). In these embodiments, the method generally includes storing the correlation data for the first and second aliquots in an identical data file in the data storage medium.

In some embodiments, the method includes processing the second sample fraction, e.g., as part of a process of purifying and analyzing multiple compounds in a complex compound library. In these embodiments, the method includes (e) splitting the second sample fraction into at least two aliquots, which aliquots of the second sample fraction are substantially representative of one another. These embodiments also include (f) collecting at least a first aliquot of the second sample fraction in the fraction collection component, and (g) storing at least a second aliquot of the second sample fraction at least transiently in the representative sample fraction storage component. In certain embodiments, the method includes substantially simultaneously (e.g., in parallel, etc.) detecting at least one property of the first aliquots of the first and second sample fractions collected in the fraction collection component, e.g., as part of further processing steps after a series of sample fractions have been purified and analyzed. Essentially any number of sample fractions can be processed according to these methods.

### BRIEF DESCRIPTION OF THE DRAWINGS

FIG. 1 schematically illustrates a purification system according to one embodiment of the invention.

FIG. 2 is a block diagram that schematically depicts a method of storing sample fraction aliquots according to one embodiment of the invention.

FIG. 3 schematically shows a purification and analytical system according to one embodiment of the invention.

FIG. 3A schematically illustrates a parallel purification and analytical system according to one embodiment of the invention.

FIG. 4 schematically illustrates a side view of a capacity altering device where the external processing regions are contained in a holder.

FIG. 5 schematically depicts the external processing regions and open holder of the capacity altering device of FIG. 4 from a top perspective view.

FIG. 6 schematically depicts the open holder of the capacity altering device of FIG. 4 from another top perspective view.

FIG. 7 schematically shows a cross-section of a portion of the capacity altering device and holder of FIG. 4 resting on a loading support platform.

FIG. 8 schematically shows a bottom perspective view of the body structure of the capacity altering device of FIG. 4.

FIG. 9 is a block diagram that schematically shows a controller control box operably connected to system components according to one embodiment of the invention.

FIG. 10 is a circuit diagram that schematically illustrates components of the controller control box of FIG. 9.

FIG. 11 schematically shows a representative example logic device in which various aspects of the present invention may be embodied.

FIG. 12 is a block diagram illustrating a software-controlled integrated high-throughput purification process according to one embodiment of the invention.

FIG. 13 is a block diagram showing a purification process according to one embodiment of the invention.

FIG. 14A schematically depicts the system of FIG. 3 in an analytical mode.

FIG. 14B schematically shows a portion of a purification and analytical system that includes multiple representative sample fraction storage components according to one embodiment of the invention.

FIG. 14C schematically shows a portion of a purification and analytical system that includes multiple representative sample fraction storage components according to one embodiment of the invention.

FIG. 14D schematically shows a portion of a purification and analytical system that includes multiple representative sample fraction storage components according to one embodiment of the invention.

FIG. 14E schematically shows a portion of a purification and analytical system that includes multiple representative sample fraction storage components according to one embodiment of the invention.

FIG. 15 is a graph that illustrates an exemplary pump method in percentage of mobile phase. The abscissa of the graph represents the percentage, while the ordinate represents time (minutes).

FIG. 16 is a graph that shows another exemplary pump method. The abscissa of the graph represents the flow rate (mL/minutes) and the ordinate represents time (minutes).

FIG. 17 schematically depicts the system of FIG. 3 in a preparative mode.

FIG. 18 schematically depicts the system of FIG. 3 in a collection mode.

FIG. 19 schematically depicts the system of FIG. 3 in an injection mode.

FIG. 20 schematically depicts the system of FIG. 3 in a quality control (QC) mode.

FIG. 21 schematically depicts the system of FIG. 3 in a post-QC mode.

FIGS. 22A-C are graphs that show pre-purification QC data. In particular, FIG. 22A is a total ion chromatogram (TIC) in which the abscissa of the graph represents intensity, while the ordinate represents the time (minutes). FIG. 22B is an extracted ion chromatogram (XIC) in which the abscissa of the graph represents intensity, while the ordinate represents the time (minutes). FIG. 22C is a graph of ELSD data in which the abscissa of the graph represents intensity and the ordinate represents the time (minutes).

FIGS. 23A-C are graphs that show preparatory-purification QC data. In particular, FIG. 23A is a total ion chromatogram (TIC) in which the abscissa of the graph represents intensity, while the ordinate represents the time (minutes). FIG. 23B is an extracted ion chromatogram (XIC) in which the abscissa of the graph represents intensity, while the ordinate represents the time (minutes). FIG. 23C is a graph of ELSD data in which the abscissa of the graph represents intensity and the ordinate represents the time (minutes).

FIGS. 24A-C are graphs that show post-purification QC data. In particular,

FIG. 24A is a total ion chromatogram (TIC) in which the abscissa of the graph represents intensity, while the ordinate represents the time (minutes). FIG. 24B is an extracted ion chromatogram (XIC) in which the abscissa of the graph represents intensity, while the ordinate represents the time (minutes). FIG. 24C is a graph of ELSD data in which the abscissa of the graph represents intensity and the ordinate represents the time (minutes).

FIG. 25 is an ELSD calibration curve done for samples before purification (with no internal standard correction). The abscissa of the graph represents average raw area and the ordinate represents mg of sample injected.

FIG. 26 is an ELSD calibration curve done for samples before purification (with internal standard correction). The abscissa of the graph represents average raw area and the ordinate represents mg of sample injected.

FIG. 27 is a graph of ELSD data of samples in purification (i.e., in prep-QC). In particular, the ELSD plot was constructed using QC data obtained using representative sample fraction aliquots. The abscissa of the graph represents average raw area, while the ordinate represents mg injected.

FIG. 28 is a graph of ELSD data of samples from collection (i.e., ELSD of Thr). The abscissa of the graph represents average raw area and the ordinate represents 5% of the sample fraction (in mg), approximately the relative amount transiently stored in the second fraction of the first sample injected.

FIG. 29 is a graph that provides an overlay of ELSD signals from prep-QC and post-QC analyses. The abscissa of the graph represents average raw area and the ordinate represents mg sample injected.

### DETAILED DESCRIPTION

I. Definitions

Before describing the present invention in detail, it is to be understood that this invention is not limited to particular embodiments. It is also to be understood that the terminology used herein is for the purpose of describing particular embodiments only, and is not intended to be limiting. Units, prefixes, and symbols are denoted in the forms suggested by the International System of Units (SI), unless specified otherwise. Numeric ranges are inclusive of the numbers defining the range. Further, unless defined otherwise, all technical and scientific terms used herein have the same meaning as commonly understood by one of ordinary skill in the art to which the invention pertains. The terms defined below, and grammatical variants thereof, are more fully defined by reference to the specification in its entirety.

The term “bottom” refers to the lowest point, level, surface, or part of a device or system, or device or system component, when oriented for typical designed or intended operational use.

The term “communicates” in the context of detection components refers to the positioning, configuration, or orientation of those components for the detection of detectable signals or properties that they are designed to detect. In some embodiments, for example, detection components are positioned downstream from one or more chromatographic components to detect detectable signals of or from sample components after those components have been separated from one another by the chromatographic components.

The term “corresponding” in the context of representative sample fraction aliquots and sample fractions refers to a representative sample fraction aliquot and a sample fraction that have been split or otherwise separated from one another.

The term “fluid communication” or “fluidly communicate” in the context of dispensing system components refers to the ability of fluids (e.g., liquids, supercritical fluids, etc.) to be conveyed between those components.

The term “representative sample fraction aliquot” or “proportional sample fraction aliquot” refers to a portion of a sample fraction.

The term “substantially representative aliquot of a sample fraction” refers a portion of a sample fraction that includes at least approximately the same composition as the sample fraction. In certain embodiments, for example, a substantially representative aliquot of a sample fraction is split away or otherwise taken from the sample fraction as the sample fraction is collected in a fraction collection component. Substantially representative aliquots of sample fractions are typically at least transiently stored in representative sample fraction storage components prior to being analyzed to provide a purity assessment of the corresponding collected sample fraction.

The term “top” refers to the highest point, level, surface, or part of a device or system, or device or system component, when oriented for typical designed or intended operational use, such as positioning object storage modules, storing objects, and/or the like.

II. Overview

While the present invention will be described with reference to a few specific embodiments, the description is illustrative of the invention and is not to be construed as limiting the invention. Various modifications can be made to the embodiments of the invention described herein by those skilled in the art without departing from the true scope of the invention as defined by the appended claims. It is also noted here that for a better understanding, certain like components are designated by like reference letters and/or numerals throughout the various figures.

The present invention relates generally to compound purification and analysis. In certain embodiments, for example, the systems and methods described herein provide the ability to sample a representative sample fraction aliquot from a sample fraction as it is collected and to analyze that aliquot immediately after the purification or preparative chromatographic run is completed for the sample fraction. This provides post-purification QC data within the same run as purification for the sample fraction. These approaches to fraction purity assessment provide for faster sample processing than pre-existing methods, e.g., by reducing post-purification sample handling (e.g., liquid handling, collected fraction agitation or evaporation and reconstitution, etc.) that is characteristic of these other techniques. In some embodiments, the systems and methods described herein perform a purity assessment or QC run or analysis whether or not a sample fraction is collected. In addition to these high-throughput methods and systems for performing sample purification and analysis, related computer program products are also provided that can also be used to significantly facilitate the purification and analysis of, e.g., libraries in production and/or being screened.

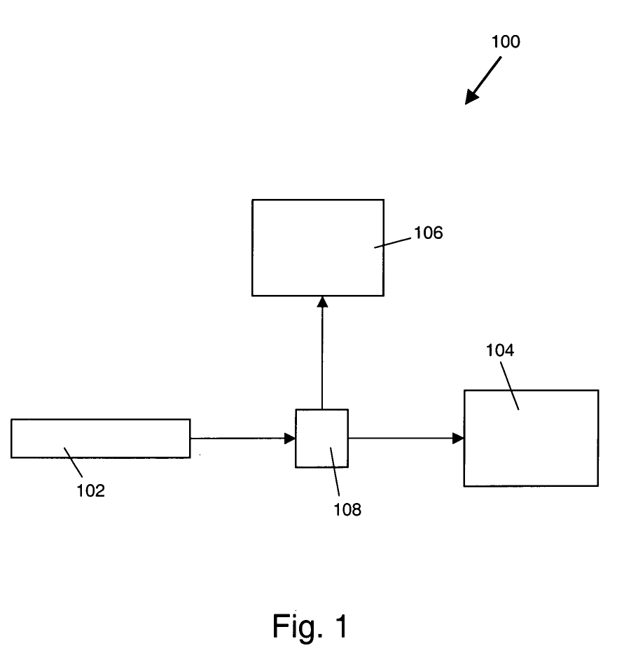

As an overview, FIG. 1 schematically illustrates a purification system according to one embodiment of the invention. As shown, system 100 includes preparative chromatographic component 102, which is configured to separate sample components from one another. Although not shown, system 100 also typically includes a handling component that is configured to convey samples into an inlet of preparative chromatographic component 102. System 100 also includes fraction collection component 104 that is structured to collect sample fractions. In addition, system 100 also includes representative sample fraction storage component 106 that is structured to at least transiently store representative sample fraction aliquots. In some embodiments, for example, representative sample fraction storage components include fluid loops or other fluid channels that are at least partially disposed in one or more switching valves of the purification systems. System 100 also includes splitting mechanism 108 that fluidly communicates with preparative chromatographic component 102, fraction collection component 104, and representative sample fraction storage component 106. Splitting mechanism 108 is structured to split (e.g., actively or passively) sample fractions flowed toward fraction collection component 104 such that substantially representative aliquots of the sample fractions are also flowed to representative sample fraction storage component 106. Typically, the purification systems described herein also include analytical chromatographic components in fluid communication with representative sample fraction storage components such that representative sample fraction aliquots stored in the representative sample fraction storage components can analyzed immediately after the corresponding sample fractions are collected in fraction collection components. In other aspects, the invention provides system software that effects collection and transient storage of representative sample fraction aliquots in the purification systems described herein.

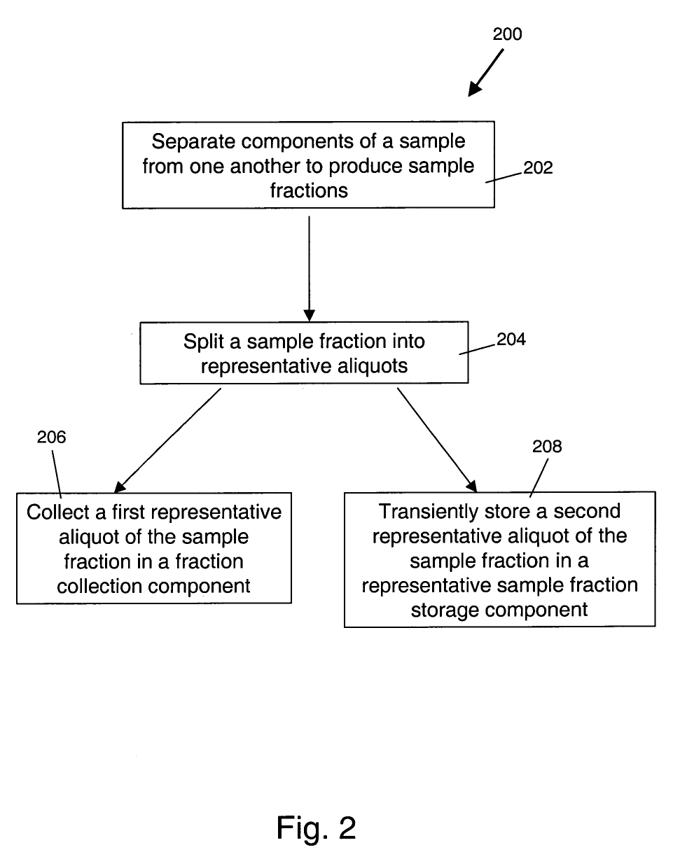

To further illustrate, FIG. 2 is a block diagram that schematically depicts a method of storing sample fraction aliquots according to one embodiment of the invention. As shown, method 200 includes separating components of a sample from one another to produce sample fractions in step 202, and splitting a sample fraction into at least two representative aliquots in step 204. As shown in step 206, method 200 also includes collecting a first representative aliquot of the sample fraction in a fraction collection component. In addition, method 200 also includes transiently storing a second representative aliquot of the sample fraction in a representative sample fraction storage component in step 208. Steps 206 and 208 are generally performed substantially simultaneous with one another, e.g., as the sample fraction is flowed through a splitting mechanism. Typically, the second representative aliquot stored in the representative sample fraction storage component is rapidly analyzed to provide, e.g., purity data that is correlated with the corresponding first representative aliquot of the sample fraction collected in the fraction collection component. This process significantly improves throughput relative, e.g., to pre-existing techniques that analyze sample fractions collected in fraction collection components instead of transiently stored representative aliquots. These and a variety of additional features of the present invention will become evident upon complete review of this disclosure, including the examples provided below.

III. Purification and Analytical Systems

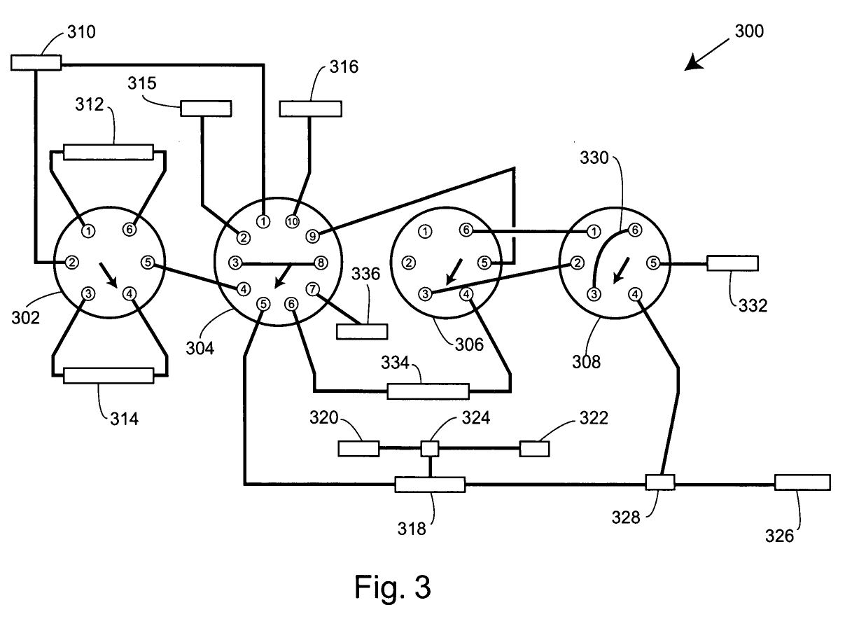

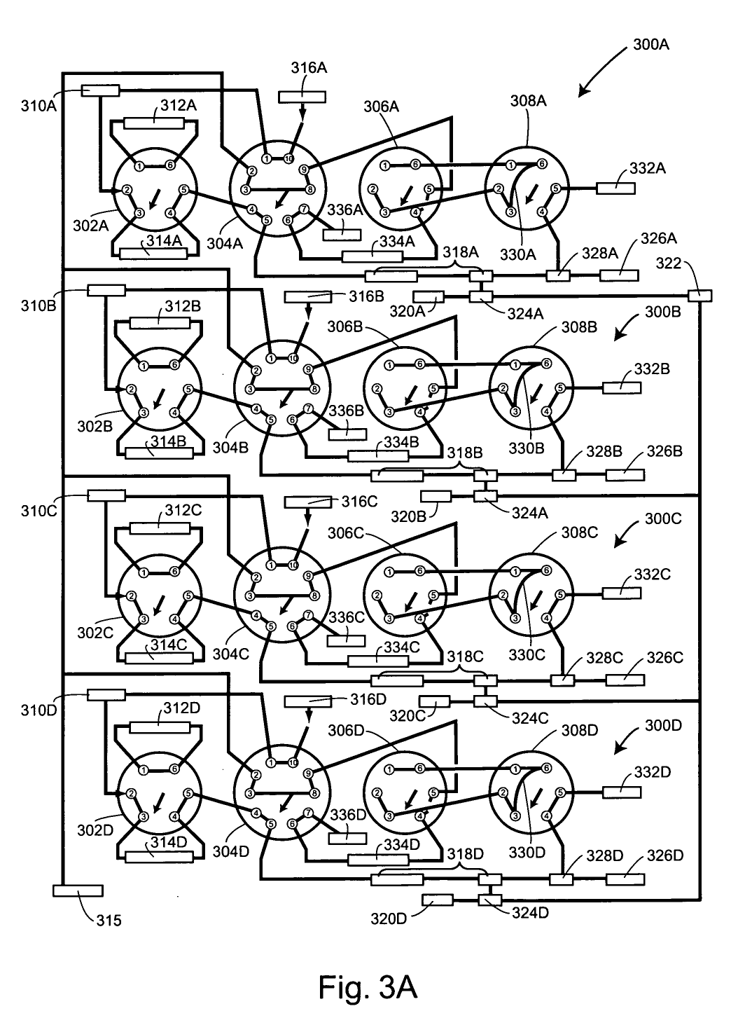

To further illustrate, FIG. 3 schematically shows a purification and analytical system according to one embodiment of the invention. As shown, system 300 includes four switching valves 302, 304, 306, and 308. Switching valves 302, 306, and 308 are shown as six port valves, while switching valve 304 is depicted as a 10 port valve. Handling component 310 (e.g., an autosampler, etc.) fluidly communicates (e.g., via conduits) with switching valves 302 and 304 via ports 2 and 1, respectively. Handling component 310 is configured to selectively convey samples into analytical chromatographic component 312 or preparative chromatographic component 314. Pump 316 (e.g., a binary pump) can fluidly communicate with handling component 310 via ports 10 and 1 of switching valve 304. Pump 315 can fluidly communicate with port 1 or 3 of switching valve 304 via port 2 of switching valve 304. Pump 315 can be used, for example, as a re-equilibration pump. Analytical chromatographic component 312 fluidly communicates with switching valve 302 via ports 1 and 6, while preparative chromatographic component 314 fluidly communicates with switching valve 302 via ports 3 and 4. Analytical chromatographic component 312 and preparative chromatographic component 314 can each fluidly communicate with switching valve 304 (via port 4 thereof) via port 5 of switching valve 302.

Detection component 318 (e.g., a UV detector fluidly coupled to a splitting mechanism, etc.) communicates with port 5 of switching valve 304 and can selectively fluidly communicate with port 4 or 6 of switching valve 304. In some embodiments, a splitting mechanism that is not fluidly coupled to a detector is included in place of detection component 318. Detection component 318 also fluidly communicates with detection component 320 (e.g., an evaporative light-scattering detector, etc.) and detection component 322 (e.g., a mass spectrometer, etc.) via splitter 324. In addition, detection component 318 also fluidly communicates with fraction collection component 326 and port 4 of switching valve 308 (via splitting mechanism 328), which can selectively communicate with representative sample fraction storage component 330 via, e.g., port 3 of switching valve 308. Although not shown, a waste component typically fluidly communicates with fraction collection component 326. Detection component 318 can also fluidly communicate with waste component 332 via ports 4 and 5 of switching valve 308. As shown, representative sample fraction storage component 330 is illustrated as a fluid loop or conduit disposed between ports 3 and 6 of switching valve 308. In other embodiments, representative sample fraction storage components are disposed between other ports (e.g., in conduits disposed between other or additional ports of switching valve 308, in conduits disposed between ports of switching valves 306 and 308, etc.).

During operation, representative aliquots of sample fractions that flow toward fraction collection component 326 for collection are split away from the sample fractions and flowed into representative sample fraction storage component 330. When representative aliquots of sample fractions are analyzed they are typically flowed from representative sample fraction storage component 330 through analytical chromatographic component 334 via, e.g., ports 3 and 4 of switching valve 306. In certain embodiments, for example, pump 316 effects the flow of representative aliquots of sample fractions through analytical chromatographic component 334 via a flow path through ports 10 and 9 of switching valve 304, ports 5 and 6 of switching valve 306, ports 1, 6, 3, and 2 of switching valve 308, and ports 3 and 4 of switching valve 306. Analytical chromatographic component 334 selectively fluidly communicates with ports 7 and 5 of switching valve 304 via port 6 of switching valve 304. When analytical chromatographic component 334 selectively fluidly communicates with port 7 of switching valve 304, flow from analytical chromatographic component 334 is directed to waste component 336. When analytical chromatographic component 334 selectively fluidly communicates with port 5 of switching valve 304, flow from analytical chromatographic component 334 is directed to detection component 318. Among the advantages of this component configuration is that representative aliquots of sample fractions flowed through analytical chromatographic component 334 are flowed to the same set of detection components (i.e., detection components 318, 320, and 322) to which corresponding sample fractions were previously flowed prior to collection. This eliminates a potential source of bias that may otherwise result if different sets of detection components were used at these points of detection. In some embodiments, multiple systems (e.g., multiple systems 300) are run parallel with one another, e.g., to further increase throughput of a particular purification and analysis process. For example, 2, 3, 4, 5, 6, 7, 8, 9, 10, or more systems 300, each having its own fraction collection component 326, can be run as a parallel purification and analytical system. To further illustrate, FIG. 3A schematically depicts a parallel, four-channel version of purification and analytical system 300 according to one embodiment of the invention. Letters following numerical labels in FIG. 3A denote parallel system components. For example, systems 300A, 300B, 300C, and 300D refer to the four parallel sub-systems of the overall system. In certain embodiments, a Purification Factory™ (Waters Corporation (Milford, Mass., USA)) is utilized as a starting point in the system. Other exemplary parallel systems are referred to herein.

A wide variety of switching valves can be used in the systems described herein. Although manual switching valves are optionally utilized, automated switching valves are generally included in the systems described herein, e.g., to enhance throughput and to facilitate sample processing. Different switching valve formats are commercially available and can be adapted for use in the systems of the invention. For example, switching valves can have various numbers of ports disposed in valve stators, e.g., 2, 3, 4, 5, 6, 7, 8, 9, 10, or more ports. In addition, switching valves can vary according to the number of positions or alternative flow paths into which they can be switched. For example, two-, three-, four-, six-, or other position switching valves are optionally utilized. To illustrate, system 300, described above, includes three six-port, two position switching valves (i.e., switching valves 302, 306, and 308) and one 10-port, two position switching valve (i.e., switching valve 304).

Suitable switching valves are commercially available from various suppliers including, e.g., Rheodyne LLC (Rohnert Park, Calif., USA) and Valco Instruments Co. Inc. (Houston, Tex., USA). In certain embodiments, for example, Rheodyne LabPRO™ PR700-102-01, 10-port two position, Rheodyne LabPRO™ EV700-100, 6-port two position, and Vici EHMA 6-port two position switching valves are used. Switching valves are also described in, e.g., U.S. Pat. No. 5,803,117, entitled “MULTI-ROUTE FULL SWEEP SELECTION VALVE” issued Sep. 8, 1998 to Olsen et al., which is incorporated by reference.

Conduits and fittings are typically selected according to the solvent or other reagent and pressure conditions to which they are to be exposed. Exemplary materials used to fabricated conduits (e.g., tubing, etc.) and/or fittings (e.g., nuts, ferrules, etc.) include fluorinated ethylene propylene (FEP), polytetrafluoroethylene (PTFE) (TEFLON™), perfluoroalkoxy (PFA), autoprene, C-FLEX® (a styrene-ethylene-butylene (SEBS) modified block copolymer with silicone oil), NORPRENE® (a polypropylene-based material), PHARMED® (a polypropylene-based material), silicon, TYGON®, VITON®, TEFZEL® (includes a range of fluoropolymer elastomers), polypropylene, polystyrene, polysulfone, polyethylene, polymethylpentene, polydimethylsiloxane (PDMS), polycarbonate, polyvinylchloride (PVC), polymethylmethacrylate (PMMA), polyetheretherketone (PEEK™), and the like. Conduits and/or fittings are also optionally fabricated from other materials including glass and various metals (e.g., stainless steel, etc.). Conduits and fitting are available from many different commercial suppliers including, e.g., Rheodyne LLC (Rohnert Park, Calif., USA) (e.g., RheFlex® fittings and tubing), Valco Instruments Co. Inc. (Houston, Tex., USA), and the like. Moreover, although larger sizes are optionally utilized, cavities disposed through conduits typically include, e.g., cross-sectional dimensions of between about 10 μm and about 10 mm, more typically between about 100 μm and about 5 mm, and still more typically between about 500 μm and about 1 mm. In some embodiments, for example, conduit cavities include inner diameters, such as 0.0025″, 0.004″, 0.005″, 0.006″, 0.007″, 0.010″, 0.015″, 0.020″, 0.030″, 0.040″, or the like.

Although other configurations are optionally utilized, the representative sample fraction storage components of the systems described herein are typically formed at least in part in switching valves and conduits, e.g., in the form of a fluid loop. Representative sample fraction storage components are used to transiently store representative sample fraction aliquots prior to analysis. Representative sample fraction storage component 330 in FIG. 3 schematically depicts one embodiment of a representative sample fraction storage component used in the systems of the invention. Representative sample fraction storage components are typically designed in view of the volumes of representative sample fraction aliquots that they are to store prior to analysis. As described further below, these volumes are dependent on the volumes of sample fractions collected in fraction collectors in certain embodiments. In some embodiments, for example, splitting mechanisms are configured to split at a set ratio (e.g., 1:20) of the fraction being collected. To illustrate, representative sample fraction storage components typically have volume capacities of between about 100 μL and about 10 mL (e.g., about 200 μL, about 300 μL, about 400 μL, about 500 μL, about 600 μL, about 700 μL, about 800 μL, about 900 μL, etc.), more typically have volume capacities of between about 1 mL and about 8 mL, and still more typically have volume capacities of between about 2 mL and about 6 mL. In some embodiments, representative sample fraction storage component volume capacities can be adjusted by storing representative aliquots in longer or shorter fluid pathways in the particular system.

The purification and analysis systems of the invention include splitting mechanisms that split sample fractions as they are flowed toward the fraction collection component such that substantially representative aliquots of the sample fractions are flowed to the representative sample fraction storage component for storage pending further analysis. Splitting mechanisms are typically configured to split sample fractions such that representative aliquots of sample fractions are flowed to representative sample fraction storage components at set ratios. Exemplary split ratios that are optionally utilized include, e.g., 1:1, 1:2, 1:5, 1:10, 1:20, 1:40, and the like. Splitting mechanisms that are optionally included in the systems described herein are available from various commercial suppliers including, e.g., SGE, Inc. (Austin, Tex., USA), BASi (West Lafayette, Ind., USA), and LC Packings (Sunnyvale, Calif., USA).

Separated sample fractions are generally collected using fraction collectors. Suitable fraction collectors are typically capable of handling a wide variety of collection vessels, such as microtiter plates and tubes (e.g., test tubes, vials, microcentrifuge tubes, mini tubes, etc.). Exemplary types of fraction collectors that can be adapted for use in the systems described herein include fraction collectors having rotatably mounted turntables for supporting multiple collection vessels and fraction collectors in which collection vessels are arranged in rectangular grid patterns. Sample fractions are typically sequentially dispensed into the collection vessels through opening in conduits (e.g., hollow needles or dispensing tips) that are mounted on arms that can position the conduit openings over each collection vessels. Fraction collectors are also described in, e.g., U.S. Pat. No. 6,450,218, entitled “FRACTION COLLECTOR,” which issued Sep. 17, 2002 to Andersson, U.S. Pat. No. 6,280,627, entitled “LIQUID CHROMATOGRAPH WITH FRACTION COLLECTOR,” which issued Aug. 28, 2001 to Kobayashi, U.S. Pat. No. 4,862,932, entitled “FRACTION COLLECTOR,” which issued Sep. 5, 1989 to Feinstein et al., and U.S. Pat. No. 5,541,420, entitled “MULTI-SAMPLE FRACTION COLLECTOR BY ELECTROPHORESIS,” which issued Jul. 30, 1996 to Kambara, which are each incorporated by reference. Moreover, suitable fraction collectors are readily available from various commercial suppliers including, e.g., Gilson, Inc. (Middleton, Wis., USA), Bio-Rad Laboratories (Hercules, Calif., USA), Shimadzu Corporation (Kyoto, Japan), Eldex Laboratories, Inc. (Napa, Calif., USA), and Amersham Biosciences Corp. (Piscataway, N.J., USA). In certain embodiments, for example, a Gilson FC 204 Fraction Collector is utilized.

Multi-well containers that are optionally utilized as collection vessels include those having, e.g., 6, 12, 24, 48, 96, 192, 384, 768, 1536, or more wells. Many of these are available from various commercial suppliers including, e.g., Greiner America Corp. (Lake Mary, Fla., USA), Nalge Nunc International (Rochester, N.Y., USA), H+ P Labortechnik AG (Oberschleiβheim, Germany), and the like. Suitbable test tubes, vials, and associated racks are also readily available from various suppliers known in the art including many of those referred to herein.

Fraction collection components utilized in the systems described herein optionally include holders (e.g., racks that have collections sites (e.g., containers)) and capacity altering devices (e.g., or tube adapters) in some embodiments. These devices facilitate sample handling, e.g., by reducing post-purification sample handling. In some embodiments, for example, holders can contain at least one capacity altering device or a portion thereof (e.g., sample tubes or a multi-well plate). The holders can, for example, be configured to allow centrifugation of a device contained or partially contained in the holder and/or can comprise features that minimize the amount of handling (e.g., of sample tubes) required during use of such a device. In addition, capacity altering devices are particularly useful in processing samples whose volume exceeds the capacity of external sample processing regions (e.g., sample tubes or wells). Holders and capacity altering devices are also described in, e.g., International Publication No. WO 2004/034026, entitled “CAPACITY ALTERING DEVICE, HOLDER AND METHODS OF SAMPLE PROCESSING,” filed Oct. 8, 2003 by Backes et al., and U.S. Application Publication No. U.S. 2004/0096986, entitled “PHARMACEUTICAL COMBI-CHEM PURIFICATION FACTORY SYSTEM,” filed Nov. 13, 2003 by Klein et al., which are both incorporated by reference.

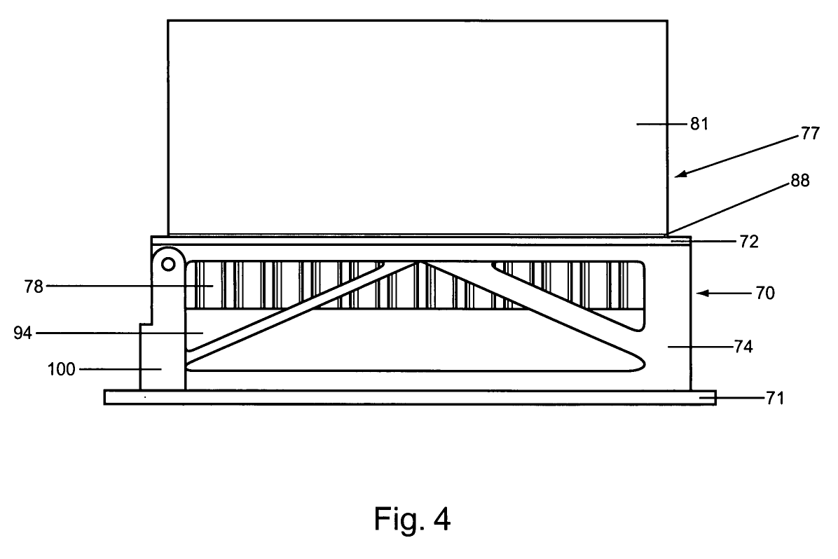

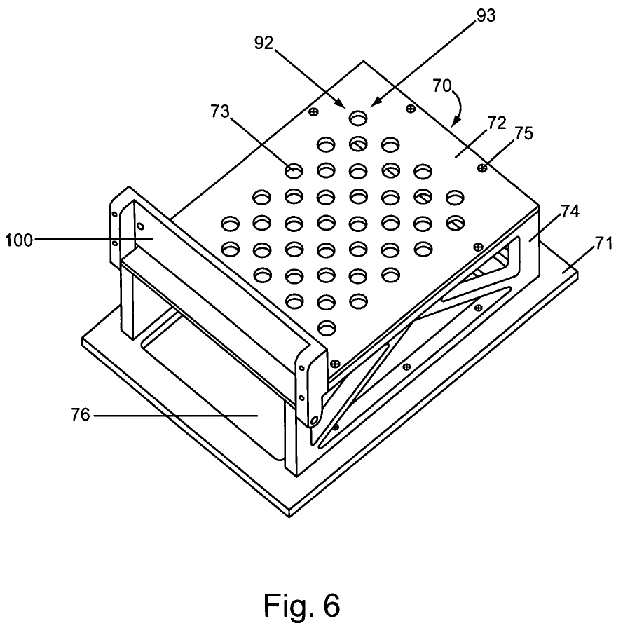

To further illustrate, one class of exemplary embodiments is illustrated in FIGS. 4-7. In this class of embodiments, holder 70 comprises base 71, top plate 72 comprising, e.g., forty-eight apertures 73, and a coupling mechanism comprising three partial side walls 74. Side walls 74 permanently couple top plate 72 to base 71 in a first fixed position. As depicted, screws 75 (e.g., stainless steel screws) attach each side wall 74 to base 71 and top plate 72. Holder 70 in the first fixed position is configured to be inserted in a centrifuge carrier. Body structure 81 with extensions 90 and sample tubes 78 comprises capacity altering device 77. In this class of embodiments, holder 70 contains, e.g., forty-eight sample tubes 78 that comprise forty-eight external processing regions 79. (As depicted, holder 70 contains an additional forty-eight unused sample tubes 78.) Body structure 81 is disposed on top plate 72, such that top plate 72 is between body structure 81 and sample tubes 78. As depicted, body structure 81 is in contact with top plate 72, and top plate 72 is in contact with sample tubes 78, but this need not be the case in other embodiments. Body structure 81 comprises forty-eight first access apertures 82, forty-eight inner cavities 84 comprising internal processing regions 85, and forty-eight second access apertures 83. In some embodiments, body structures include sloping regions disposed between internal processing regions and second access apertures. These sloping regions typically slope inwards from the walls of internal processing regions towards second access apertures by between about 0° and about 90°, more typically by between about 15° and about 75°, and still more typically by between about 25° and about 65° (e.g., about 30°, about 35°, about 40°, about 45°, about 50°, etc.). To illustrate, sloping region 86 of body structure 81 exemplifies one of these embodiments. Sloping regions direct collected fractions towards second access apertures.

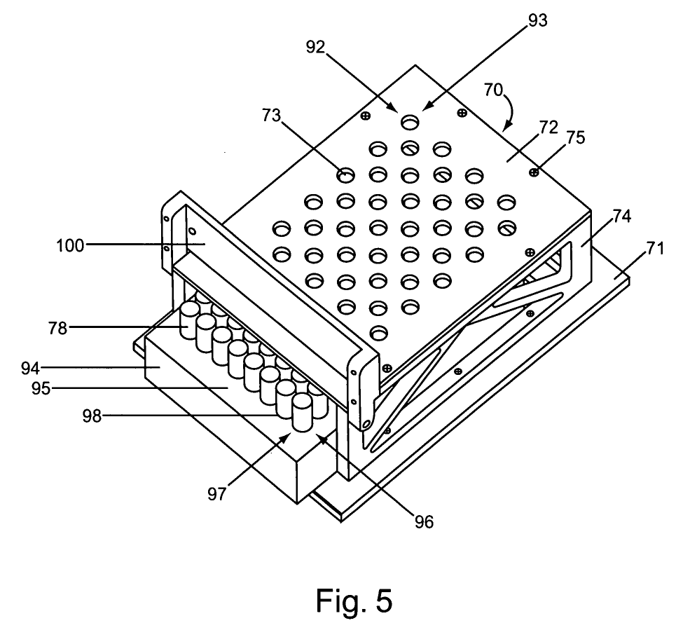

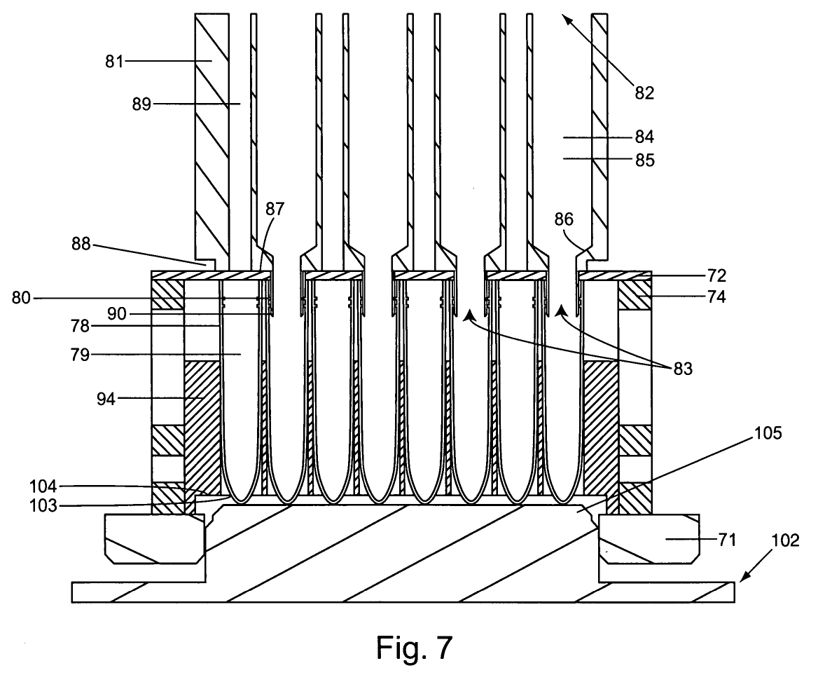

As depicted, body structure 81 comprises, e.g., forty-eight cavities 89, which decrease the weight of body structure 81 but which need not be present in other embodiments. Body structure 81 is removably sealed with, e.g., forty-eight sample tubes 78 such that internal processing regions 85 are removably sealed to external processing regions 79. The forty-eight apertures 73 in the top plate are spatially arranged (in twelve staggered columns 92 of four apertures 73 and eight rows 93 of six apertures 73) to correspond to the positions of second access apertures 83. Sample tubes 78 are positioned in tube rack 94. As shown, tube rack 94 has ninety-six apertures 98 in top surface 95 (arranged in 12 columns 96 and eight rows 97, corresponding to the wells of a ninety-six well multi-well plate), although only alternate tubes are accessible through apertures 73 in top plate 72. Tube rack 94 and sample tubes 78 can, e.g., be purchased from Matrix Technologies Corp. (Hudson, N.H., USA). Body structure 81 is removably sealed to sample tubes 78 by forty-eight extensions 90 projecting from bottom surface 87 of body structure 81 through apertures 73. Extensions 90 form pressed, radial seals with sample tubes 78. Sample tubes 78 as purchased from Matrix Technologies Corp. each comprise two radial protrusions 80 that form removable seals with extensions 90. Tubes lacking such protrusions can also be used. The diameter of apertures 73 in top plate 72 is less than the outer diameter of the top of sample tubes 78. Body structure 81 can thus be, e.g., lifted up off holder 70, e.g., by inserting a small pry bar (e.g., a screwdriver) into groove 88 and prying body structure 81 off holder 70, to detach extensions 90 from sample tubes 78, thereby uncoupling internal processing regions 85 from external processing regions 79, while sample tubes 78 are retained in holder 70. Handling of sample tubes 78 is thus minimized. As depicted, holder 70 comprises door 100, which can be opened as shown in FIG. 5 to allow sample tubes 78 and tube rack 94 to be positioned in or removed from holder 70, or closed as shown in FIG. 4 to secure tube rack 94 in holder 70. Holder 70 need not comprise a door, since tube rack 94 can be secured in holder 70 merely by coupling body structure 81 with sample tubes 78. As depicted in this class of example embodiments, base 71 comprises rectangular aperture 76. The presence of aperture 76 decreases the weight of holder 70, but is not necessary; thus, in other embodiments, the base of the holder is, e.g., solid or comprises more than one aperture. Tube rack 94 as purchased from Matrix Technologies Corp. comprises ninety-six apertures 103 in its bottom surface 104. Removably sealing body structure 81 with sample tubes 78 can involve the exertion of force (e.g., of about 50 pounds) on body structure 81 and sample tubes 78; in some instances, this force can be sufficient to displace tubes 78 through apertures 103. Temporary placement of, e.g., loading support platform 102 under holder 70 prior to sealing body structure 81 to sample tubes 78 can prevent such displacement of tubes 78. As depicted in FIG. 7, sample tubes 78 rest on raised portion 105 of loading support platform 102, which raised portion 105 projects upward into aperture 76 in base 71 of holder 70.

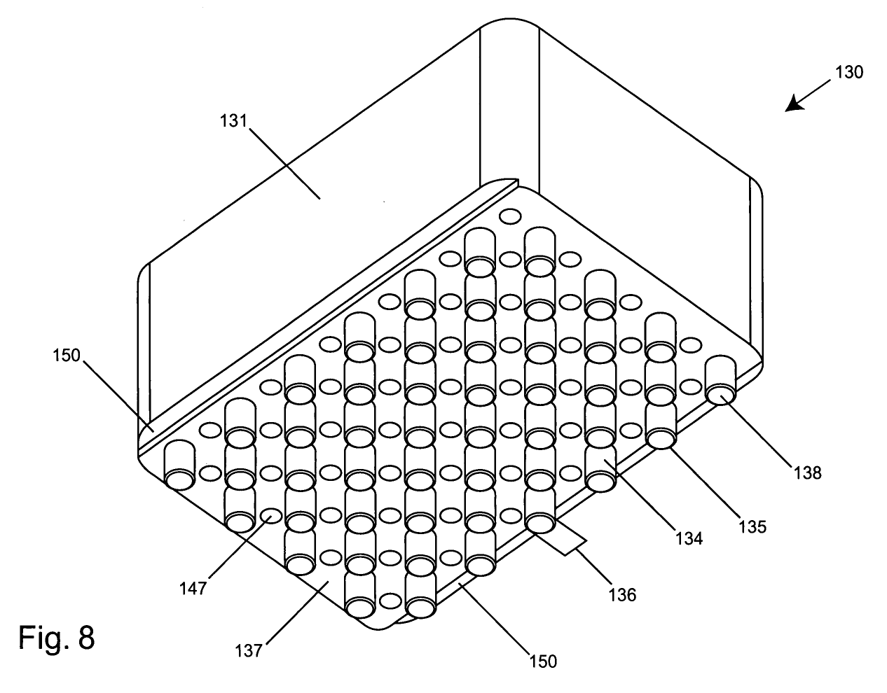

Yet another class of embodiments is illustrated in FIG. 8. In this class of embodiments, capacity altering device 130 comprises body structure 131, forty-eight sample tubes (not shown), and a sealing mechanism that comprises forty-eight straight extensions 134 projecting from bottom surface 137 of body structure 131. Body structure 131 comprises forty-eight first access apertures (located in a top surface of body structure 131 and arranged in twelve staggered columns of four first access apertures and eight rows of six first access apertures) connected to and separated from forty-eight second access apertures 138 by forty-eight inner cavities. The inner cavities comprise forty-eight internal processing regions (not shown). Each extension 134 has terminus 135 at which one of circular second access apertures 138 is located. Outer diameter 136 of a cross-section of each extension 134 is essentially constant along the extension, from near body structure 131 to terminus 135 of the extension. Extensions 134 form pressed, radial seals with external processing regions comprising sample tubes. Grooves 150 (depicted as, e.g., a groove running along each of two edges of bottom surface 137 of body structure 131) can facilitate removal of body structure 131 from sample tubes. As depicted, body structure 131 comprises forty-eight cavities 147 parallel to inner cavities. Cavities 147 reduce the weight of body structure 131 but need not be present in all embodiments.

Chromatographic components used to separate sample components from one another (e.g., components of representative sample fraction aliquots). Essentially any chromatographic component can be adapted for use in the systems described herein. In some embodiments, for example, chromatographic components are chromatography columns, e.g., liquid chromatography columns, supercritical fluid chromatography columns, or the like. In liquid chromatography (LC) (including, high performance liquid chromatography (HPLC)), the mobile phase is liquid, while the stationary phase is, e.g., a liquid adsorbed on a solid (e.g., in liquid-liquid or partition chromatography), an organic species bonded to a solid surface (e.g., in liquid-bonded phase chromatography), solid (e.g., in liquid-solid or adsorption chromatography), ion exchange resin (e.g., in ion exchange chromatography), polymeric solids (e.g., in size exclusion chromatography), or the like. In supercritical-fluid chromatography (SFC), the mobile phase is a supercritical fluid and the stationary phase is, e.g., an organic species bonded to a solid surface. Supercritical fluids have properties that are intermediate between liquids and gases, and exist above supercritical temperatures and pressures for the particular substance.

Chromatography is well-known to those of skill in the art and is also described in, e.g., Grob et al. (Eds.), Modern Practice of Gas Chromatogaphy, 4^{th} Ed., John Wiley & Sons, Inc. (2004), Ardrey, Liquid Chromatography—Mass Spectrometry: An Introduction, John Wiley & Sons, Inc. (2003), Brown et al., Advances in Chromatography Vol. 42, Marcel Dekker (2003), and Williams et al. (Eds.), Supercritical Fluid Methods and Protocols, Vol. 13, Methods in Biotechnology Series, Humana Press (2000), which are each incorporated by reference. In addition, chromatography columns, related instrumentation, and consumables are available from various commercial suppliers including, e.g., Peeke Scientific (Redwood City, Calif., USA), Waters Corporation (Milford, Mass., USA), Valco Instruments Co. Inc. (Houston, Tex., USA), Essential Life Solutions Ltd. (Boston, Mass., USA), BioChrom Labs, Inc. (Terre Haute, Ind., USA), Polymer Standards Service GmbH (Mainz, Germany), and PerkinElmer Life and Analytical Sciences, Inc. (Boston, Mass., USA). To further illustrate, in some embodiments of system 300 schematically shown in FIG. 3, analytical chromatographic components 312 and 334 are C18-Q, C18, 5 μm, 50×4.6 mm analytical chromatography columns, and preparative chromatographic component 314 is an Ultro 120 C18 U1-5C18-ME, 5 μm, 50×10 mm preparative chromatography column, which are each available from Peeke Scientific (Redwood City, Calif., USA).

In some embodiments, the chromatographic components included in the systems described herein are electrophoretic devices. To illustrate, capillary electrophoresis instrumentation is optionally adapted for use in the systems described herein. Capillary electrophoresis (CE) is a family of related separation techniques that use narrow-bore fused-silica capillaries to separate a complex array of large and small molecules. High voltages are used to separate molecules based on differences in charge, size and hydrophobicity. Injection into the capillary is typically accomplished by immersing the end of the capillary into a sample vial and applying pressure, vacuum or voltage. Commercial suppliers of CE-related instrumentation and consumables include, e.g., Combisep, Inc. (Ames, Iowa, USA), Beckman Coulter, Inc. (Fullerton, Calif., USA), and the like.

Handling components (e.g., autosamplers, etc.) are used to convey or inject samples into the systems described herein for chromatographic separations. In certain embodiments, for example, Gilson 215 Liquid Handlers/Injectors (Gilson, Inc. Middleton, Wis., USA) are used in the systems of the invention.

The systems described herein typically include one or more pumps or other devices that are structured to convey fluids through various flow paths of the systems. Many different pumps that can be used in the systems of the invention are commercially available, such as LC-8A Shimadzu pumps (Shimadzu Corporation (Kyoto, Japan)). In some embodiments, three LC-8A Shimadzu pumps are used in the systems described herein.

Controllers are typically operably connected to one or more system components, such as switching valves, fraction collection components, handling components, pumps, detection components, fluid sensors, or the like, to control operation of these components. More specifically, controllers are generally included either as separate or integral system components that are utilized, e.g., to effect the conveyance of samples into chromatographic components for separation, the pump flow rates, the detection and/or analysis of detectable signals received from sample components by detectors, etc. Controllers and/or other system components is/are optionally coupled to an appropriately programmed processor, computer, digital device, or other logic device or information appliance (e.g., including an analog to digital or digital to analog converter as needed), which functions to instruct the operation of these instruments in accordance with preprogrammed or user input instructions (e.g., sample input volumes, flow path selections, gradient selections, etc.), receive data and information from these instruments, and interpret, manipulate and report this information to the user.

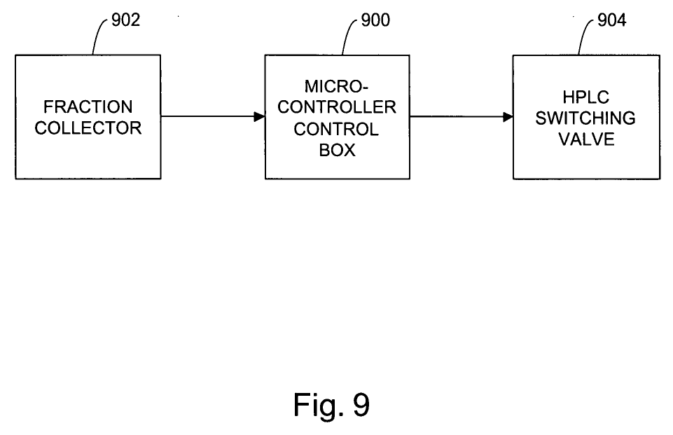

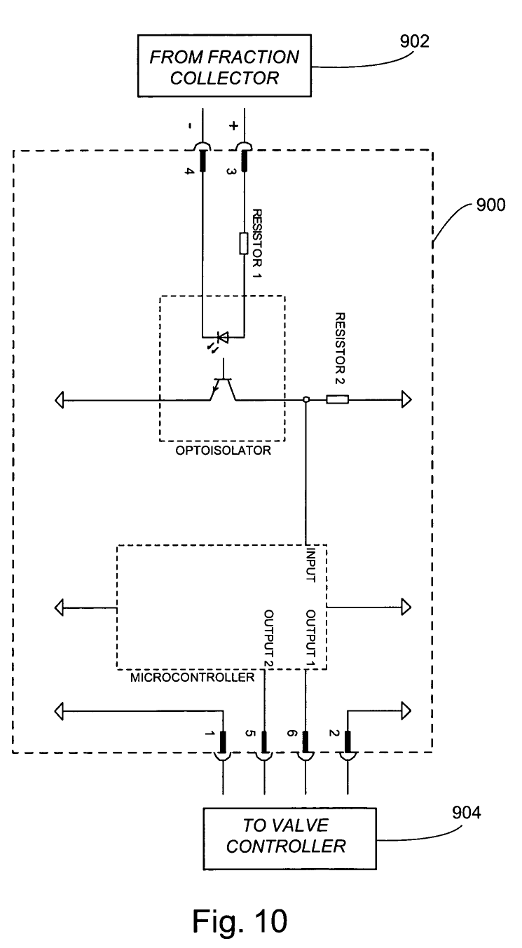

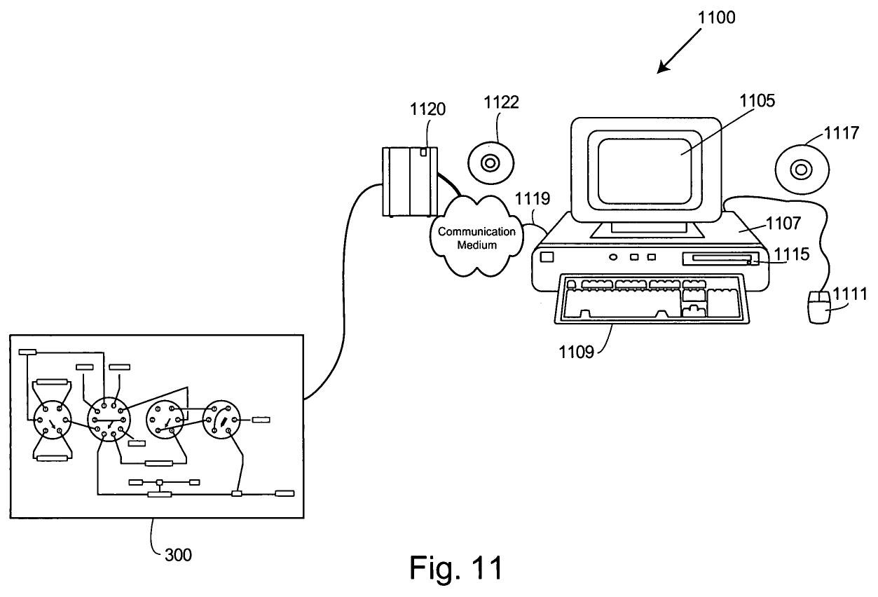

A controller or computer optionally includes a monitor, which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. To illustrate, FIG. 9 is a block diagram that schematically shows controller control box 900 operably connected to fraction collection component 902 and to switching valve 904 according to one embodiment of the invention. FIG. 10 schematically shows a diagram of circuits in controller control box 900, which effects coordination of the timing of the collection trigger of the flow sampling device or splitting mechanism and the fraction collections valve. While the timing coordination of the collection trigger could be handled using discrete logic, the microcontroller implementation illustrated in FIG. 10 has a number of advantages. For example, the microcontroller can be programmed to identify command input signals from any number of different systems (e.g. fraction collectors) and provide any type of output signal to command any type of valve controller. Additionally, the use of a microcontroller allows potentially complex switching protocols (e.g. adding a time delay, performing multiple switch events per command or the like) to be implemented easily merely by changing its software code. The command input to the microcontroller is optically isolated so there can be no unwanted electrical interaction between the various systems. Another advantage of the implementation illustrated in FIG. 10 is that no external power supply is required to run the control box. All power that is used to operate the circuitry within the control box is derived from the fraction collector and the valve controller. This minimizes system wiring, makes retrofitting the control box to legacy systems much easier, eliminates the need for batteries, and results in a small, efficient physical package. In certain embodiments, control box 900 is implemented such that it allows the operator to introduce a time delay that is adjustable from, e.g., about 0.1 seconds to about 1 second in 0.1 second increments via, e.g., a dial type, 10 position selector switch or another type of switching mechanism. The box also optionally includes a hard disk drive, a floppy disk drive, a high capacity removable drive such as a writeable CD-ROM, and other common peripheral elements. Inputting devices such as a keyboard or mouse optionally provide for input from a user. An exemplary system comprising a computer is schematically illustrated in FIG. 11.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting fluid flow rates or modes of analysis, directing collection of sample fractions, or the like. The computer then receives the data from, e.g., sensors/detectors included within the system, and interprets the data, either provides it in a user understood format, or uses that data to initiate further controller instructions, in accordance with the programming, e.g., such as in monitoring detectable signal intensity or the like.

More specifically, system computers typically include logic instructions that effect, e.g., receiving one or more user input parameters selected from the group consisting of: sample input volumes, fluid flow rates, solvent selection, gradient selection, flow path selection, representative sample fraction aliquot volumes, collected sample fraction volumes, delay time between sample component detection and sample fraction aliquot collection and/or storage, at least one fraction collection component volume capacity, and output displays; tracking samples, sample fractions, and/or representative sample fraction aliquots; receiving and storing data from the system component; correlating data received from the system component with one or more sample fractions collected by the fraction collection component; processing data to produce processed data; terminating further processing of one or more sample fractions and/or another sample if processed data fails to meet one or more selected criteria; quantifying one or more sample fraction components; altering a duration of time between detection of separated sample components and triggering sample fraction collection and representative sample fraction aliquot storage; altering a duration of time between triggering sample fraction collection and triggering representative sample fraction aliquot storage; and/or, conveying samples into an inlet of the first chromatographic component.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer which is known to one of skill. Various database servers and/or software (e.g., MySQL® database server, the ActivityBase Suite of data management software (ID Business Solutions Inc., Emeryville, Calif., USA), spreadsheet software such as Microsoft Excel™, Corel Quattro Pro™, or database programs such as Microsoft Access™ or Paradox™) can be adapted to the present invention. Suitable data management products are also available from suppliers, such as Oracle Corporation (Redwood Shores, Calif., USA). Software for performing, e.g., material conveyance to selected wells of a multi-well plate, assay detection, and data deconvolution is optionally constructed by one of skill using a standard programming language such as AppleScript, Visual basic, C, C++, Perl, Python, Fortran, Basic, Java, or the like.

FIG. 11 is a schematic showing a representative example system including an information appliance in which various aspects of the present invention may be embodied. As will be understood by practitioners in the art from the teachings provided herein, the invention is optionally implemented in hardware and software. In some embodiments, different aspects of the invention are implemented in either client-side logic or server-side logic. As will also be understood in the art, the invention or components thereof may be embodied in a media program component (e.g., a fixed media component) containing logic instructions and/or data that, when loaded into an appropriately configured computing device, cause that apparatus or system to perform according to the invention. As will additionally be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

FIG. 11 shows information appliance or digital device 1100 that may be understood as a logical apparatus (e.g., a computer, etc.) that can read instructions from media 1117 and/or network port 1119, which can optionally be connected to server 1120 having fixed media 1122. Information appliance 1100 can thereafter use those instructions to direct server or client logic, as understood in the art, to embody aspects of the invention. One type of logical apparatus that may embody the invention is a computer system as illustrated in 1100, containing CPU 1107, optional input devices 1109 and 1111, disk drives 1115 and optional monitor 1105. Fixed media 1117, or fixed media 1122 over port 1119, may be used to program such a system and may represent a disk-type optical or magnetic media, magnetic tape, solid state dynamic or static memory, or the like. In specific embodiments, the aspects of the invention may be embodied in whole or in part as software recorded on this fixed media. Exemplary computer program products are described, e.g., further below. Communication port 1119 may also be used to initially receive instructions that are used to program such a system and may represent any type of communication connection. Optionally, aspects of the invention are embodied in whole or in part within the circuitry of an application specific integrated circuit (ACIS) or a programmable logic device (PLD). In such a case, aspects of the invention may be embodied in a computer understandable descriptor language, which may be used to create an ASIC, or PLD. FIG. 11 also includes system 300, which is operably connected (e.g., via one or more components of system 300) to information appliance 1100 via server 1120. Optionally, system 300 is directly connected to information appliance 1100.

The systems of the invention typically include one or more bulk property detectors and/or one or more solute property detectors. In certain embodiments, for example, systems are configured to detect and quantify absorbance, fluorescence, mass, an electrochemical property, a refractive index, conductivity, FT-IR, light scattering, optical activity, photoionization, and/or other properties of sample components and/or other system reagents. Exemplary detectors or sensors that are included in the systems described herein include, e.g., mass spectrometers, UV/Vis detectors, evaporative light-scattering detectors (ELSD), nuclear magnetic resonance (NMR) detectors, electrochemical detectors, fluorescence detectors, chemiluminescent nitrogen detectors, refractive index detectors, thermal conductivity detectors, flame ionization detectors, photoionization detectors, electron capture detectors, radiation detectors, weight scales, and the like.

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Detection components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are both incorporated by reference. In addition, devices for monitoring system fluids that can be adapted for use in the systems described herein are described in, e.g., International Publication No. WO 03/065030, entitled “FLUID HANDLING METHODS AND SYSTEMS” by Micklash II et al.

IV. Computer Program Product

The invention also provides computer program products that include computer readable media having logic instructions for controlling various system components and effecting the performance of various process steps. To illustrate, computer program products generally include logic instructions that effect collection of aliquots of sample fractions in a fraction collection component and transient storage of representative sample fraction aliquots in representative sample fraction storage components when sample fractions. Typically, computer program products include logic instructions that effects separation of sample components from one another with chromatographic components.

In some embodiments, computer program product include logic instructions that effects one or more of: receiving user input parameters, such as sample input volumes, fluid flow rates, solvent selection, gradient selection, flow path selection, delay time between sample component detection and sample fraction aliquot collection and/or storage, fraction collection component volume capacities, output displays, etc.; tracking samples, sample fractions, and/or representative sample fraction aliquots; receiving and storing data from components of the purification system; altering a duration of time between detection of separated sample components and triggering sample fraction collection and representative sample fraction aliquot storage; altering a duration of time between triggering sample fraction collection and triggering representative sample fraction aliquot storage; correlating the data received from the purification system component with sample fractions collected by the fraction collection component; or the like.

The computer program product optionally includes logic instructions that effects detection of sample fraction or aliquot properties (e.g., absorbance, mass, etc.) with detection components of the purification system. In some of these embodiments, logic instructions are included for correlating detected properties of representative sample fraction aliquots with corresponding sample fractions collected in the fraction collection component to produce correlation data. Typically, the computer program product includes logic instructions for storing this correlation data in the same data file in a data storage medium, such as a database or the like. In some embodiments, logic instructions are provided for automatically processing the correlation data to generate processed correlation data. In these embodiments, the computer program product optionally includes logic instructions that effect termination of further processing (e.g., weighing, evaporating, lyophilizing, storing, etc.) of sample fractions and/or another sample if the processed correlation data fails to meet selected criteria, such as a level of purity or the like. In addition, logic instructions that effect quantification of components of sample fractions are also typically provided. In some of these embodiments, for example, computer program products include logic instructions for altering a duration of time during which a sample fraction is collected in the fraction collection component or altering the delay time between detecting a signal and triggering fraction collection.

The computer readable medium generally includes one or more of, e.g., a CD-ROM, a floppy disk, a tape, a flash memory device or component, a system memory device or component, a hard drive, a data signal embodied in a carrier wave, or the like.

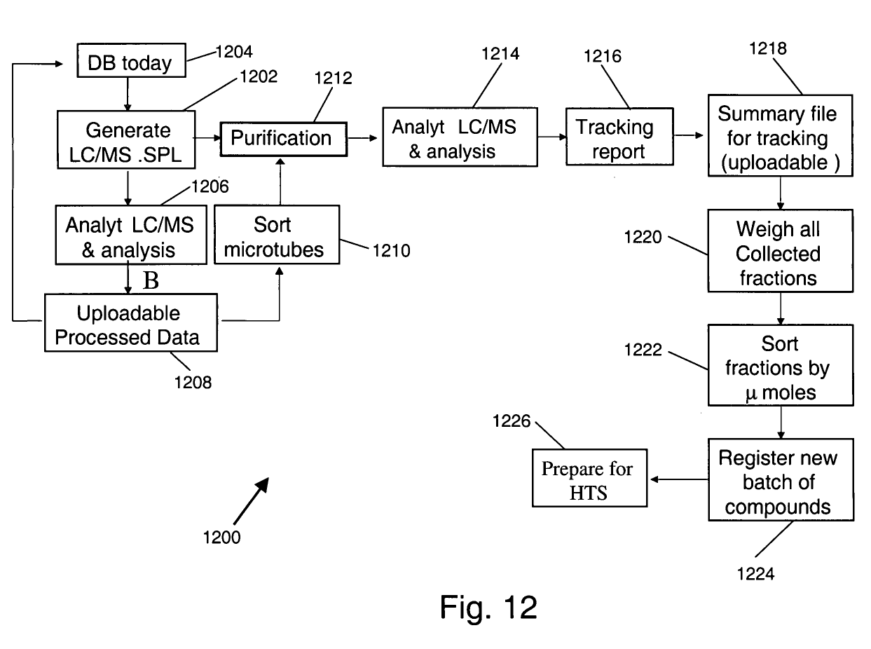

To further illustrate, FIG. 12 is a block diagram illustrating an integrated high-throughput purification process in which one or more steps may be software controlled. As shown, in method 1200 LC/MS .SPL files (step 1202), analytical LC/MS and analysis data (1206), and uploadable processed data (1208) are generated from a current database (1204). The samples submitted for purification can be sorted (step 1210) based on the results obtained from the uploadable processed data (1208). This typically is used to sort compounds prior to purification, e.g., to promote high sample processing efficiency. In some embodiments, samples are sorted using high-speed tube sorters, such as those described in, e.g., International Application No. PCT/US2003/035536, entitled “SYSTEMS AND METHODS OF SORTING SAMPLES,” filed Nov. 7, 2003 by Weselak et al., which is incorporated by reference. More specifically, the samples are typically sorted (step 1210) prior to performing a preparatory or semi-preparatory purification (step 1212), which is followed by analytical LC/MS and analysis (step 1214). From this analysis, a tracking report can be created (step 1216) and an uploadable summary file for tracking (step 1218) can be generated therefrom. The file/data from 1218 can be uploaded into database 1204. In some embodiments, steps 1212, 1214, and 1216 together comprise a single step. In addition, method 1200 includes weighing all collected fractions (step 1220), sorting fractions by μmoles (step 1222), registering a new batch of compounds (step 1224), and preparing for high throughput screening (HTS) (step 1226).

V. Purification and Analytical Methods

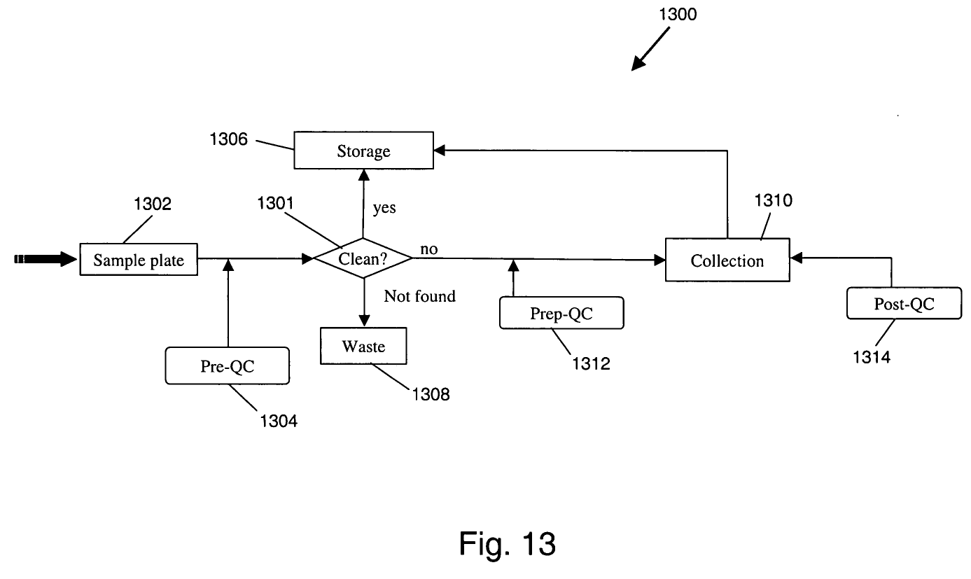

To further illustrate, FIG. 13 is a block diagram schematically showing a purification process according to one embodiment of the invention. As shown, process 1300 includes determining (step 1301) whether a sample (1302) has a desired level of purity in a pre-QC portion (1304) of the process. A sample having the desired level of purity is stored (step 1306), whereas if no sample is detected, that particular sample is sent to waste (step 1308) or storage for a later attempt using an alternative purification technique. If a sample does not have the desired level of purity, it is subjected to preparatory or semi-preparatory fluid separation and collection (step 1310) in a preparatory-QC (prep-QC) portion (1312) of the process. Following the prep-QC portion (1312), a purified sample is stored (step 1306) and a post-QC portion (1314) of the process is optionally performed. Typically, performing the prep-QC portion (1312) as described herein removes the need to perform the post-QC portion (1314). Pre-QC, prep-QC, and post-QC portions of process 1300 are each described further below and otherwise referred to herein.

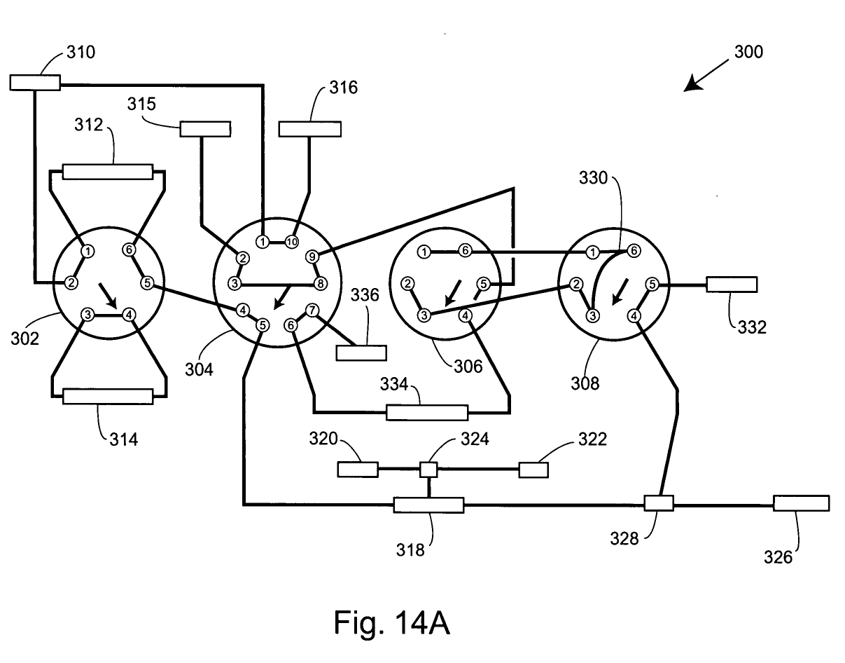

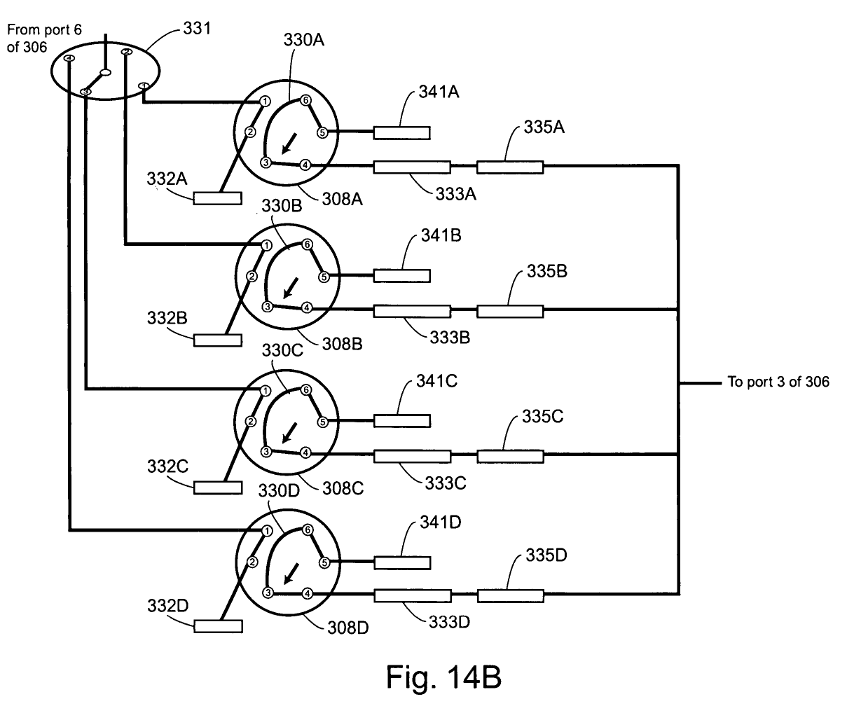

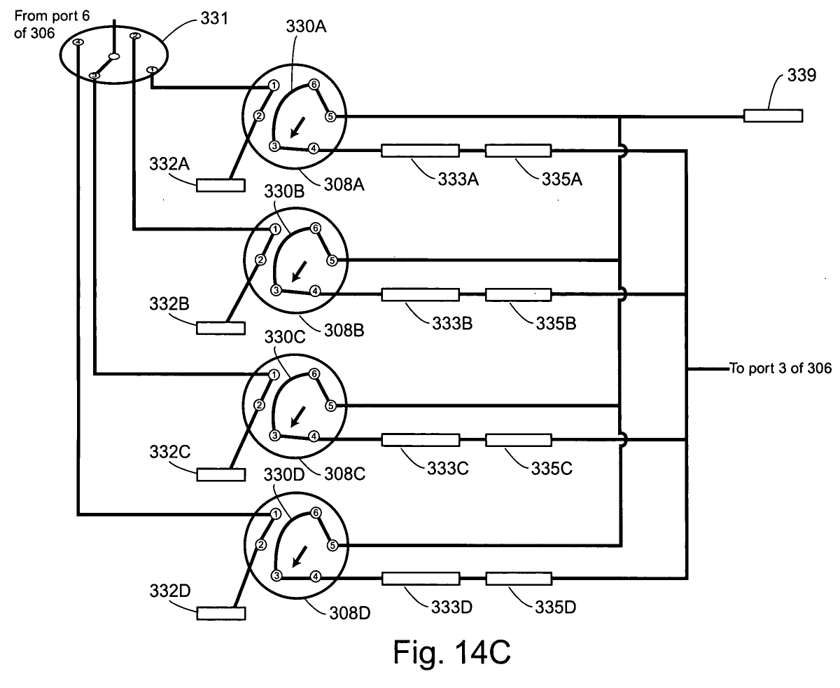

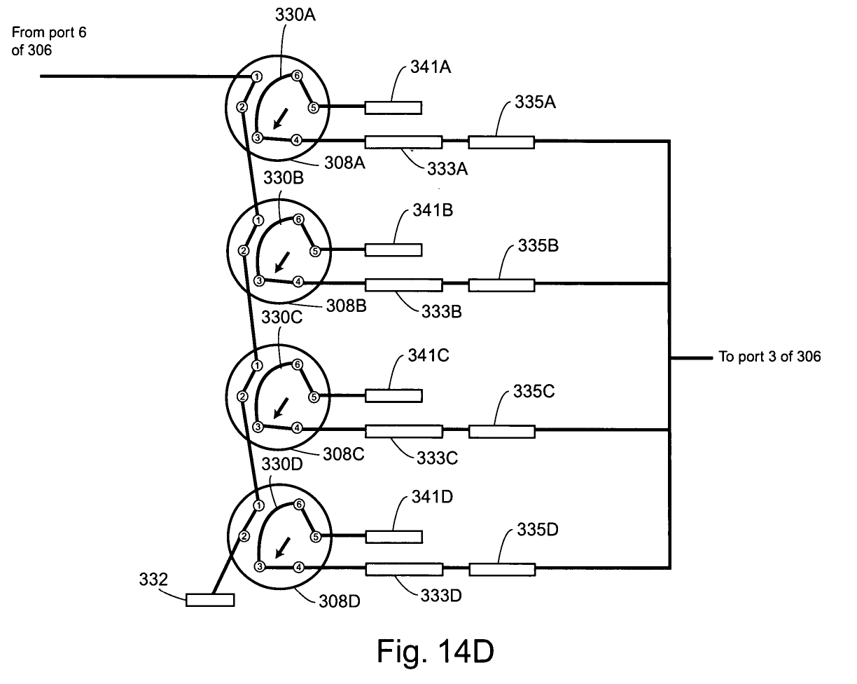

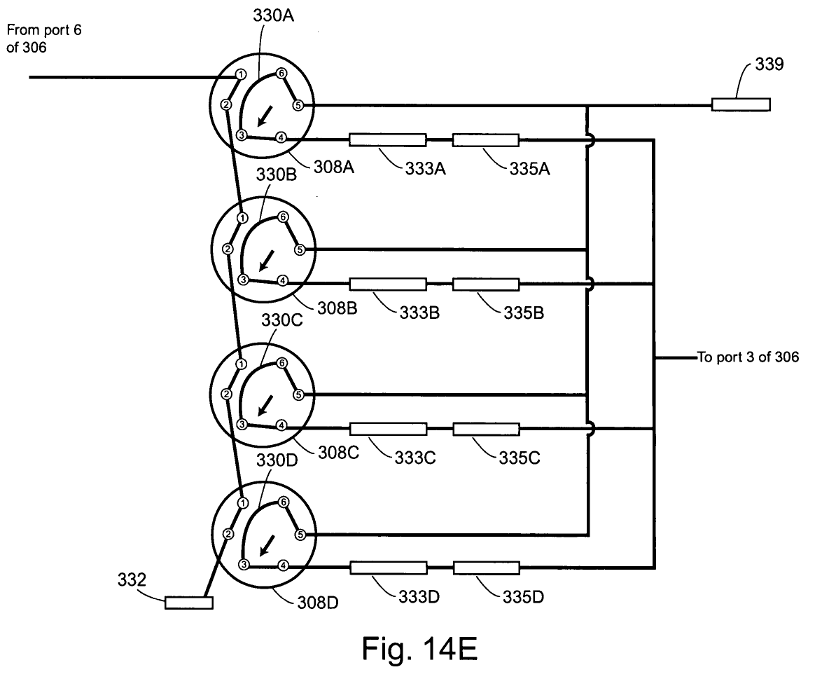

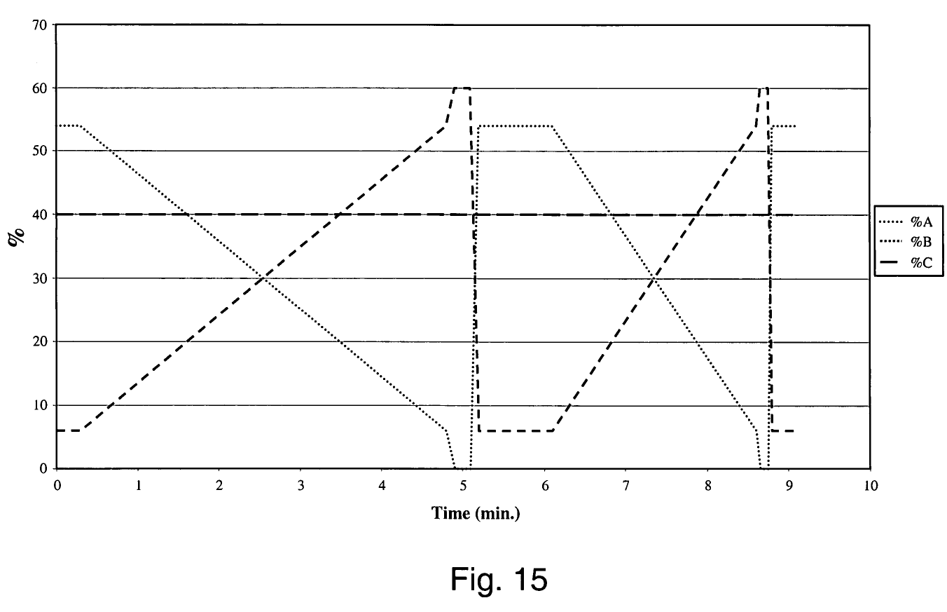

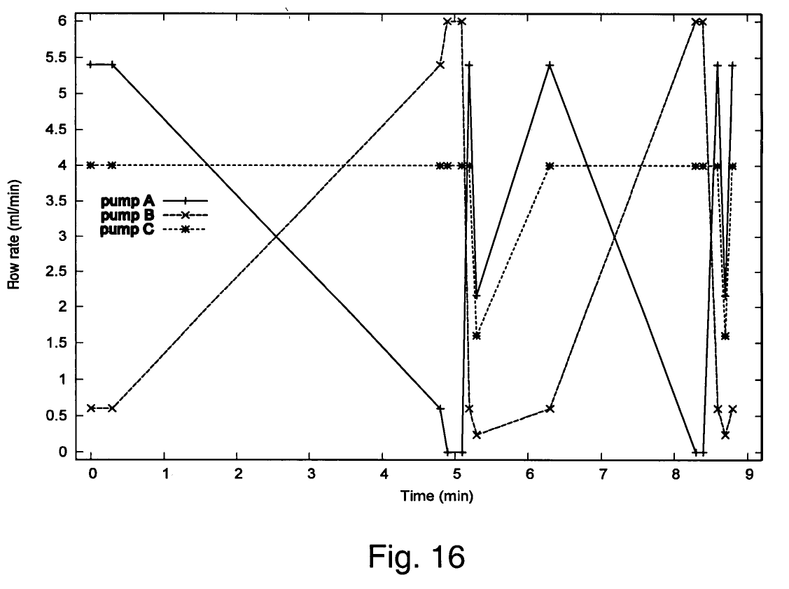

To further illustrate, FIG. 14A schematically depicts system 300 in an analytical mode that can be used to perform pre-QC and post-QC. As shown, switching valve 304 accommodates two flow paths, one used during the preparative portion (prep-QC) of a run and the other used for the analytical portion. Switching valve 302 can be used to select between these two modes, e.g., so that traditional analytical LC/MS can be performed with system 300 in an analytical mode. Further, while a gradient elution is performed using column 312, column 314 is isolated, or vice versa. Exemplary pump methods that include gradients, which can be used in the preparative and analytical modes are illustrated in FIGS. 15 and 16. Suitable eluents A, B, and C conveyed by system pumps can be readily selected by persons of skill in the art. Pump methods and eluents are also described further below in the examples.

It is to be noted that the systems and methods of the invention are not limited to transiently storing one or more representative sample fraction aliquots within the same representative sample fraction storage component. To illustrate, subsequent samples collected during one purification run may be stored in subsequent representative sample fraction storage components as schematically depicted in FIGS. 14B-E. As shown, switching valves 308A, 308B, 308C, and 308D are substituted for switching valve 308 in system 300. As shown in the embodiments of FIGS. 14 B and C, five-port, four position switching valve 331 selectively fluidly communicates with port 1 of each switching valve 308A, 308B, 308C, and 308D. The embodiments shown in FIGS. 14 D and E, which illustrate the sequential collection of multiple collections, do not include valve 331. In the embodiments depicted in FIGS. 14 B and C, port 2 of each switching valve 308A, 308B, 308C, and 308D fluidly communicates with corresponding waste components 332A, 332B, 332C, and 332D, whereas in the embodiments shown in FIGS. 14 D and E, ports 2 of switching valves 308A, 308B, 308C, and 308D sequentially fluidly communicate with waste component 332. As also shown, representative sample fraction storage components 330A, 330B, 330C, and 330D fluidly communicate with ports 3 and 6 of corresponding switching valves 308A, 308B, 308C, and 308D. Analytical chromatographic components 333A, 333B, 333C, and 333D (e.g., analytical chromatography columns, etc.) and detection components 335A, 335B, 335C, and 335D (e.g., UV detectors, etc.) fluidly communicate with port 4 of corresponding switching valves 308A, 308B, 308C, and 308D. As further shown, binary pumps 341A, 341B, 341C, and 341D (e.g., gradient HPLC pumps or the like) fluidly communicate with port 5 of corresponding switching valves 308A, 308B, 308C, and 308D in the systems schematically shown in FIGS. 14 B and D. In this configuration, switching valves 308A, 308B, 308C, and 308D can each use its own pump (e.g., for 2D chromatography using different mobile phases or gradients, etc.). In contrast, flow from binary pump 339 in the systems schematically shown in FIGS. 14 C and E is split among switching valves 308A, 308B, 308C, and 308D via port 5 of those valves. Flow from detection components 335A, 335B, 335C, and 335D is directed toward port 3 of switching valve 306.

During operation of the systems schematically depicted in FIGS. 14B-E, upon introduction of a sample into the systems with handling component 310 a signal can be sent to reset a counter, such as an operably connected information appliance 1100 as shown in FIG. 11. Upon each collection event by fraction collection component 326, the counter is typically incremented to the next integer. In the embodiments shown in FIGS. 14 B and C, the counter generally determines the port of switching valve 331 to be utilized (i.e., port 1 to switching valve 308A, port 2 to switching valve 308B, port 3 to switching valve 308C, and port 4 to switching valve 308D). In contrast, port 1 of switching valve 308A is initially utilized upon a collection event in the embodiments depicted in FIGS. 14 D and E, which can be used for the sequential collection of multiple collections, as mentioned above. When the counter has been incremented to the maximum allowable, no additional fractions or representative aliquots will typically be collected in the systems shown in FIGS. 14 B and C. Upon completion of the preparative separation, a signal can be sent to the four valves to simultaneously switch to inject in order to permit the parallel analysis of the contents of each of the four valves (i.e., the contents of representative sample fraction storage components 330A, 330B, 330C, and 330D of corresponding switching valves 308A, 308B, 308C, and 308D). During this time period, the data collected by detection components 320 and 318 can be ignored and the data collected by detection component 322 (e.g., a mass spectrometer) can be correlated with data from each of detection components 335A, 335B, 335C, and 335D (e.g., UV detectors, etc.). Essentially any other number of valves can also be adapted for use in the systems described herein, such as at least five, six, seven, eight, twelve, sixteen, or more valves. For example, the Gilson 849 and 889 injection manifolds (Gilson, Inc., Middleton, Wis., USA) consist of four and eight valves, respectively and are optionally utilized in these systems. To illustrate, an eight-channel MUX is also described in, e.g., Yan et al. (2004) “High-Throughput Purification of Combinatorial Libraries I: A High-Throughput Purification System Using an Accelerated Retention Window Approach” J. Comb. Chem. 6:255-261, which is incorporated by reference.

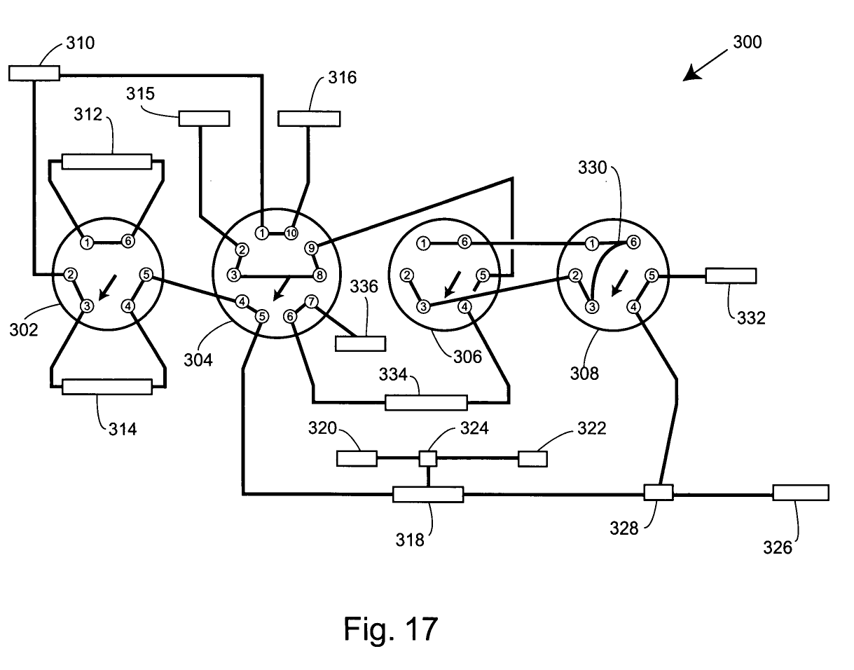

As shown in FIG. 17, system 300 is in prep-mode at the beginning of a prep-QC run. A sample injected by handling component 310 (e.g., a Gilson 215 autosampler, etc.), travels through the column selection valve (switching valve 302) to preparative chromatographic component 314 (e.g., a preparative HPLC column, a supercritical fluid column, etc.) and then to switching valve 304 and to detection components 318, 320, and 322 (e.g., UV, ELSD, and MS detectors, respectively). Typically, about 5% or less of the total flow is split evenly between detection components 320 and 322. The remaining flow is directed to fraction collection component 326 (e.g., a Gilson FC 204 fraction collector).

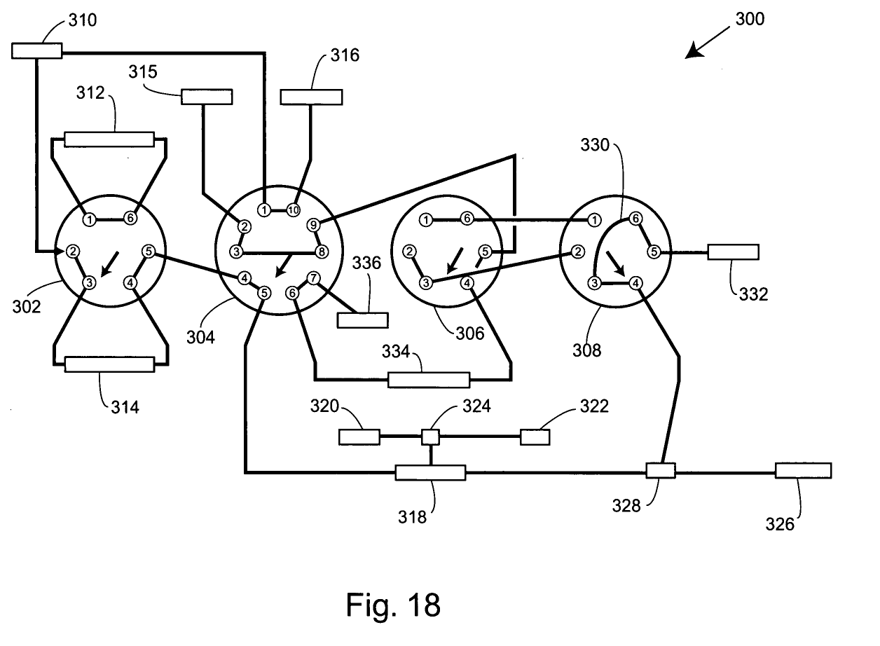

Before the collection valve of fraction collection component 326 is splitting mechanism 328 that diverts a portion from the total flow to switching valve 308. Under the control of a controller, switching valve 308 is typically synchronized with the collection valve of fraction collection component 326 so that it is switched whenever fraction collection component 326 activates its collection valve, sending the system to collection mode as shown in FIG. 18. By timing the liquid flow paths, the liquid front of a collected peak arrives at the two valves typically within about 0.1 second of each other. The proportional or representative sample fraction aliquot collected at switching valve 308 is directed into representative sample fraction storage component 330 for temporary storage until its consumption during the analytical portion of the run.

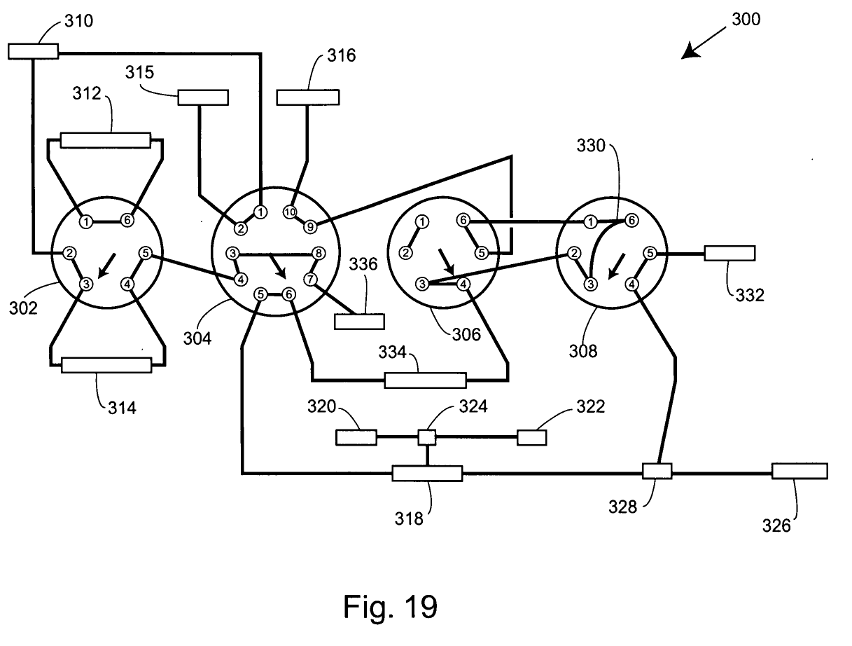

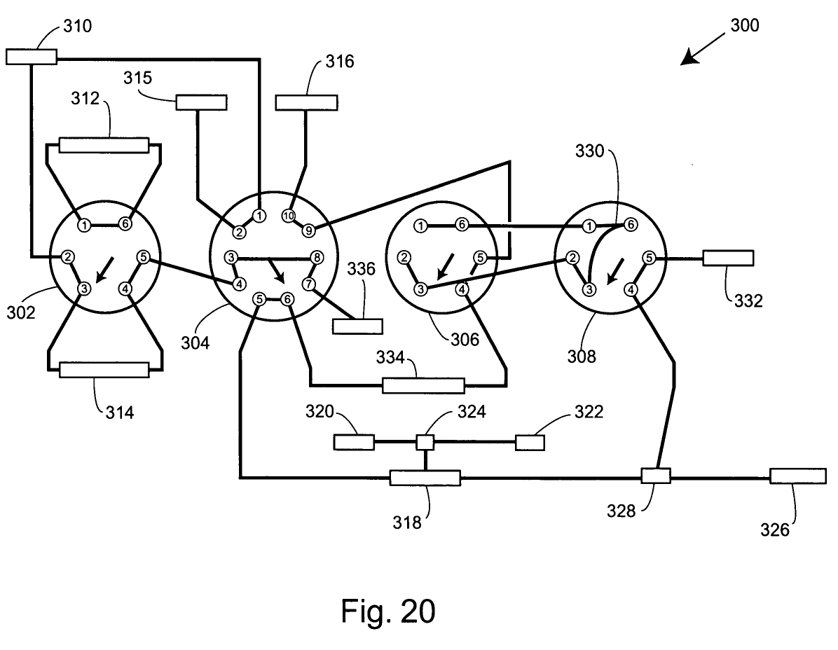

After the preparative portion of a run, the system is typically switched into a QC injection mode as shown in FIG. 19. To illustrate, switching valve 306 places representative sample fraction storage component 330 in line with analytical chromatographic component 334. After a selected period of time, system 300 goes into QC mode (shown in FIG. 20) in which switching valve 306 eliminate dwell volume and an analytical gradient begins on analytical chromatographic component 334.

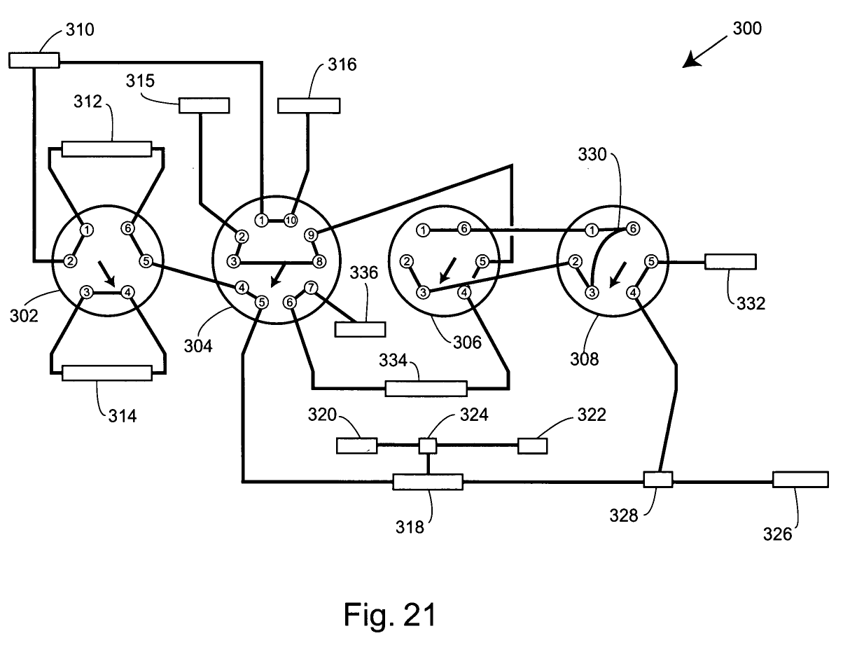

FIG. 21 schematically depicts system 300 in a post-QC mode. As referred to above, the same analytical mode is typically used for both pre-QC and post-QC (see, FIG. 14A).

### VI. EXAMPLES

The following non-limiting examples illustrate LC/MS methods for determining the purity of fractions collected immediately after preparatory fluid separations as high throughput purification processes are performed and show that aliquots stored in representative sample fraction storage components are representative of collected sample fractions.

### Example 1

### LC/MS Methods for Performing Preparative-Quality Control Cycles

This example illustrates LC/MS methods for performing preparative-quality control cycles or runs using a system described herein.

A. System Components

The system used to perform these preparative-quality control cycles had the general configuration schematically depicted in FIG. 3 and included the following commercially available components:

Hardware
3 LC-8A Shimadzu pumps
SCL-10A system controller
2 SPD-10Avp UV detectors
PE-Sciex API-150ex MS
Alltech 500 ELSD
Gilson 215 sampler
FC204 fraction collector

Valves
Rheodyne LabPRO™ PR700-102-01, 10-port two position valve
Rheodyne LabPRO™ EV700-100, 6-port two position valve
Vici EHMA 6-port two position valve

Columns
1× Semi-preparative column
Ultro 120 C18 U1-5C18-ME, 5 μm, 50×10 mm (Peeke Scientific)
2× Analytical columns
C18-Q, C18, 5 μm, 50×4.6 mm (Peeke Scientific)

Software
MassChrom 1.2.1 on a PowerMacintosh G4 (OS 9.0.4)
FC Script 2.0 to control collection.

B. Preparative-Analytical Method

In this example, eluents A and B were 0.05% trifluoroacetic acid in water and 0.035% trifluoroacetic acid (TFA) in acetonitrile. Eluent C was 0.049% TFA in 10% acetonitrile in water. The flow rate of eluent C remained constant at 4.0 mL/minute. The gradient and flow rate information is shown in FIG. 15.

Preparative LC/MS Portion

The system was in prep-mode at the beginning of the prep-QC run (see, FIG. 17). The gradient conditions included a 0.5 minute load at 10% eluent B, 10%-90% eluent B linear gradient in 4.5 min., and a 0.25 min. hold at 90% eluent B. The flow rate was 6.0 mL/min during the preparative portion of the run. The sample was injected by the Gilson 215 autosampler, traveled through the column selection valve (switching valve 302) to the preparative HPLC column (preparative chromatographic component 314) and then to switching valve 304 and to the UV, ELSD, and MS detectors (detection components 318, 320, and 322, respectively). A total of 0.20 mL/min of the total 6.0 mL/min flow was split evenly between the ELSD and the MS detectors. The remaining 5.8 mL/min flow rate was directed to a Gilson FC 204 fraction collector (fraction collection component 326).

Immediately before the collection valve of fraction collection component 326 was a 20:1 splitter (splitting mechanism 328) that diverted 0.3 mL/min from the total flow to switching valve 308. Under the control of a controller (see, FIGS. 9 and 10), switching valve 308 was synchronized with the switching valve of fraction collection component 326 so that it switched whenever fraction collection component 326 activated its switching valve, sending the system to collection mode (FIG. 18). By timing the liquid flow paths, it was found that the liquid front of a collected peak arrived at the two valves within 0.1 second of each other. The proportional or representative aliquot collected at switching valve 308 is directed into a 0.50 mL sample loop (representative sample fraction storage component 330) for temporary storage until its consumption during the analytical portion of the run (described below).

Analytical LC/MS Portion

After the preparative portion of the run, the system switched into QC injection mode (see, FIG. 19). Switching valve 306 placed the 0.50 mL aliquot collection loop in line with the analytical or QC column (analytical chromatographic component 334) for thirty seconds (3 mL). After thirty seconds, the system went into QC mode (see, FIG. 20) by switching valve 306 to eliminate the dwell volume, and the analytical gradient began on the analytical column. The analytical LC/MS portion of the run consisted of: 0.5 minute load at 10% eluent B, 10%-90% eluent B linear gradient in 3.0 min., and 0.25 min hold at 90% eluent B. The flow rate was 6.0 mL/min during the analytical portion of the run.

### Example 2

### Representative Sampling of Collected Sample Fractions

To illustrate that the representative sample fraction aliquot collected using the post purification QC configuration (described in Example 1) represented the collected fraction, calibration curves were done at the pre-purification LC/MS (e.g., to verify linearity of detector response), during preparative-QC LC/MS, and after evaporating and reconstituting the sample in 0.500 mL DMSO.

More specifically, the analysis included using a dilution series of a stock solution containing Fmoc-Thr(tBu)-OH. The total volume injected was kept constant in order to eliminate any effects due to void volumes in the injector valve system or due to uncertainties in the syringe sampling capability. To further ensure accuracy, the same concentration of Fmoc-Ala-OH was used in every sample and served as an internal standard. Stock solutions of 20 mg/mL Fmoc-Thr(tBu)-OH (Thr) and 10 mg/mL Fmoc-Ala (Ala) was prepared. Samples are made to a total of 500 μL with 80 μL of the Fmoc-Ala-OH solution, DMSO and varying volumes of the Fmoc-Thr(tBu)-OH solution to create the concentrations below in Table I.

TABLE I

| | 1 | 2 | 3 | 4 | 5 | 6 | 7 | 8 | 9 | 10 | 11 |
|---|---|---|---|---|---|---|---|---|---|---|---|
| Total | 500 | 500 | 500 | 500 | 500 | 500 | 500 | 500 | 500 | 500 | 500 |
| Volume | | | | | | | | | | | |
| (μL) | | | | | | | | | | | |
| Volume | 10 | 20 | 40 | 60 | 80 | 100 | 150 | 200 | 250 | 300 | 350 |
| of Thr | | | | | | | | | | | |
| (μL) | | | | | | | | | | | |
| Volume | 80 | 80 | 80 | 80 | 80 | 80 | 80 | 80 | 80 | 80 | 80 |
| of Ala | | | | | | | | | | | |
| (μL) | | | | | | | | | | | |
| Volume | 410 | 400 | 380 | 360 | 340 | 320 | 270 | 220 | 170 | 120 | 70 |
| of | | | | | | | | | | | |
| DMSO | | | | | | | | | | | |
| (μL) | | | | | | | | | | | |
| mg of | 0.8 | 0.8 | 0.8 | 0.8 | 0.8 | 0.8 | 0.8 | 0.8 | 0.8 | 0.8 | 0.8 |
| Ala | | | | | | | | | | | |
| mg of | 0.2 | 0.4 | 0.8 | 1.2 | 1.6 | 2 | 3 | 4 | 5 | 6 | 7 |
| Thr | | | | | | | | | | | |

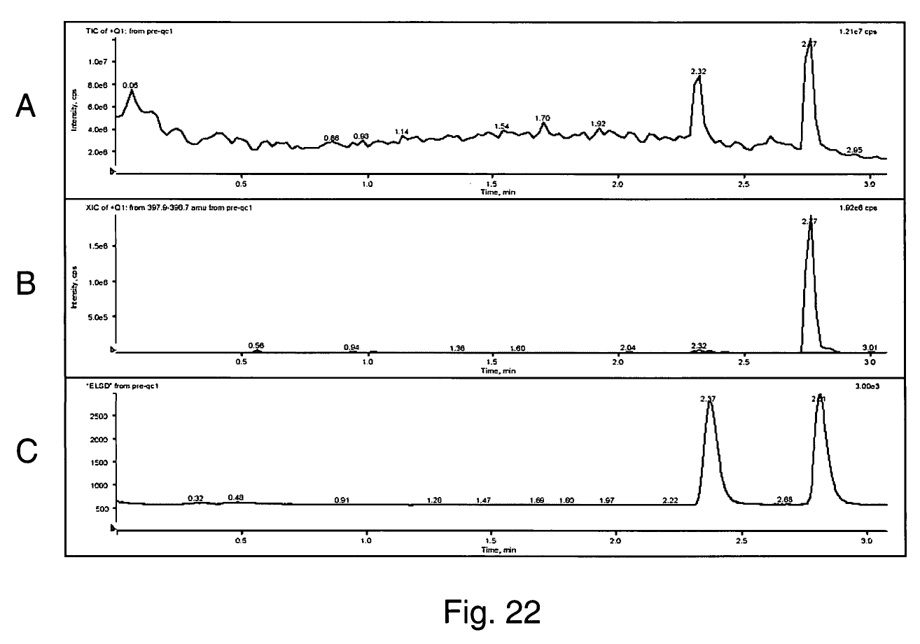

In Pre-QC mode, 25 μL, 5% of the total was injected as traditionally done for pre-purification LC/MS analysis. Typical LC/MS data is shown in FIGS. 22A-C.

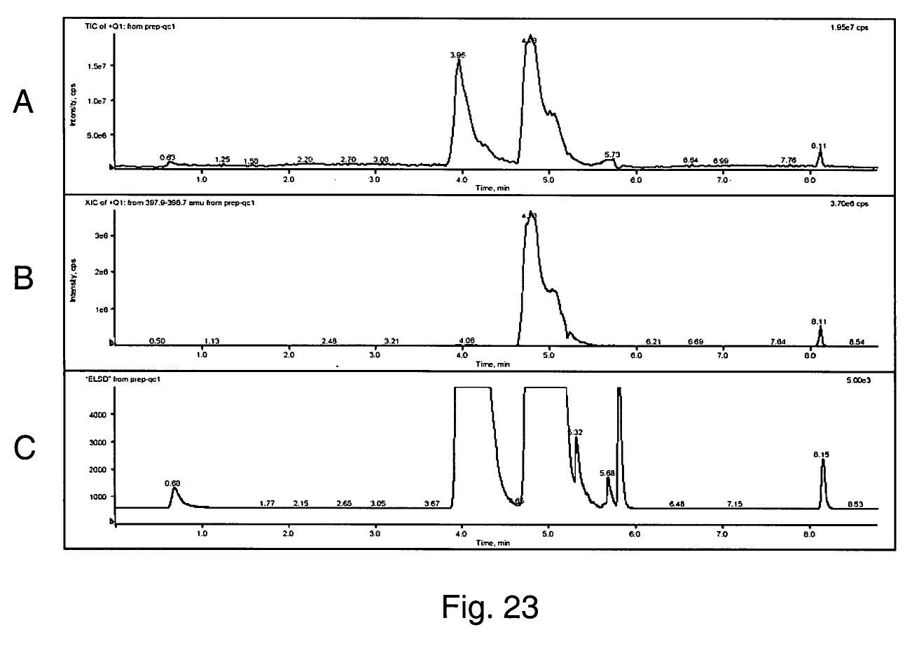

In Prep-QC mode, the remaining 475 μL was injected and the sample collected as is typically done in traditional purification. Typical LC/MS data is shown in FIGS. 23A-C. The first six minutes of the LC/MS run was the preparative portion, and the portion after six minutes was the analytical LC/MS that was generated using the representative sample fraction aliquot collected from the flow stream during the collection event and stored in the representative sample fraction storage component.

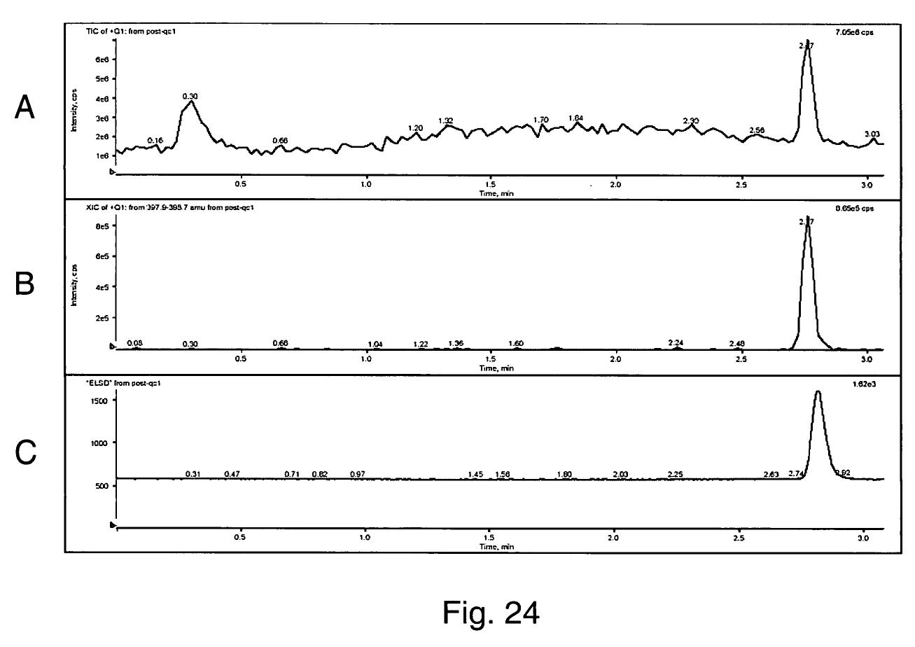

In Post-QC mode, the collection plate was dried down and DMSO was added to a total volume of 500 μL and 5% was injected to mimic representative sample fraction aliquot collection. Representative LC/MS data is shown in FIGS. 24A-C.

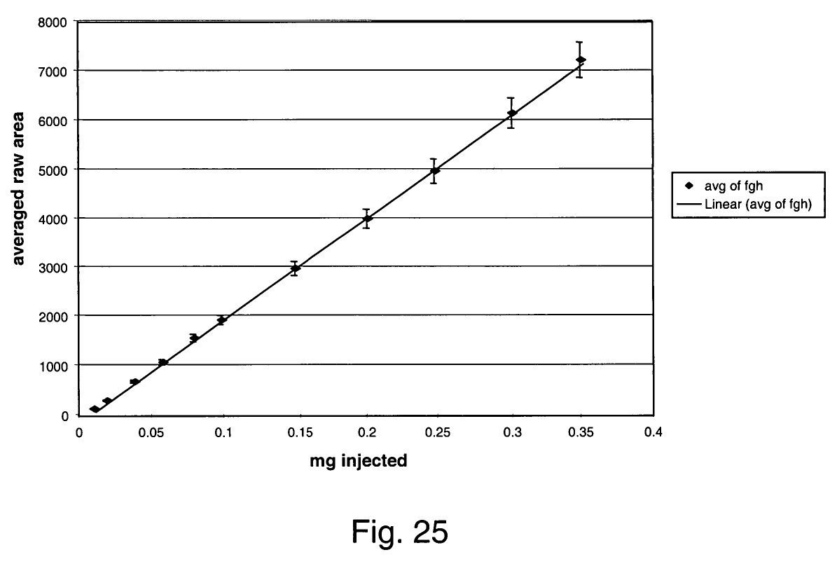

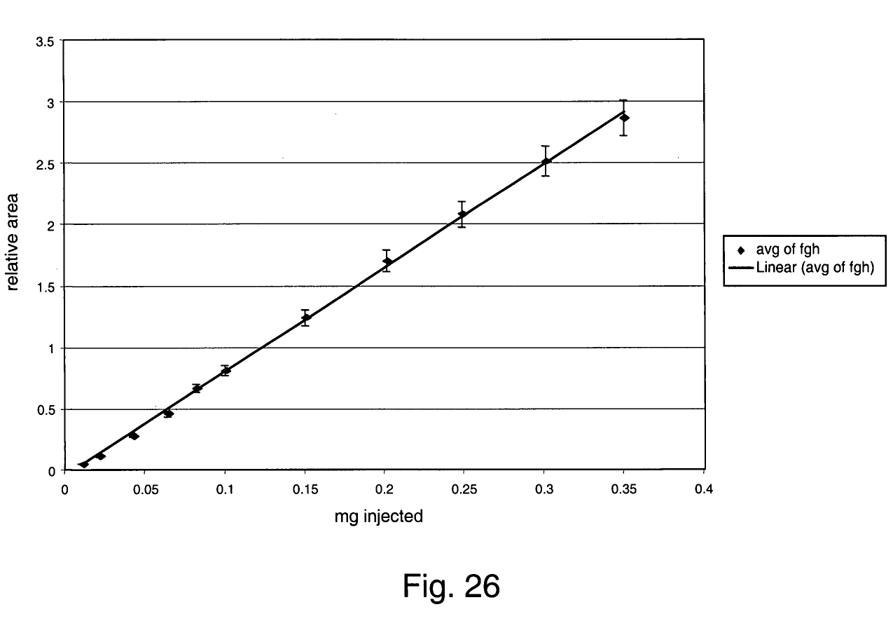

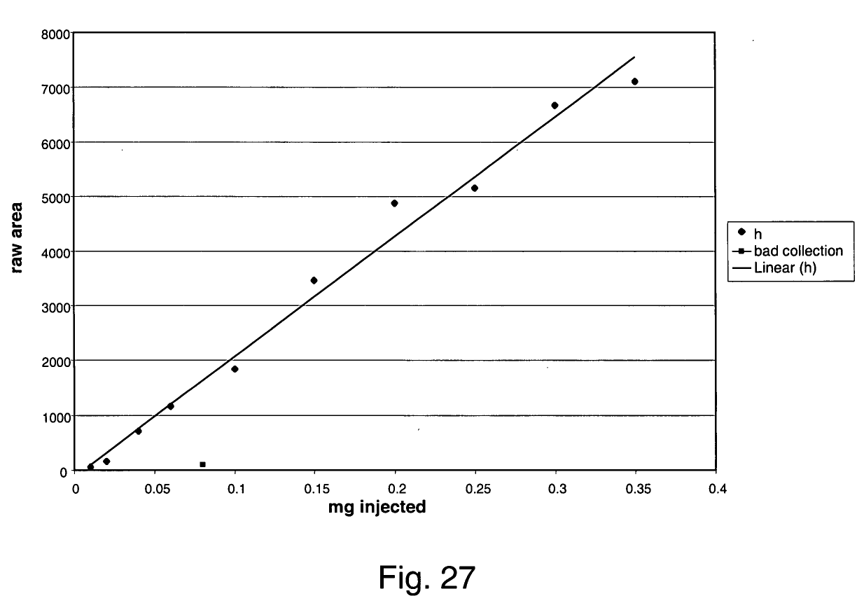

From the ELSD calibration curve done for the pre-purification samples (shown in FIG. 25 (with no internal standard correction); FIG. 26 (with internal standard correction)) it can be seen that the response was linear in the range of 0.010 to 0.35 mg injected. Since 5% of the material in each of the source wells was injected to create this calibration curve and since 5% of the collected material is sampled for the post-purification analysis portion of Prep-QC, purification of 0.2-7.0 mg of Thr was expected to provide linear response, as shown in FIG. 27.

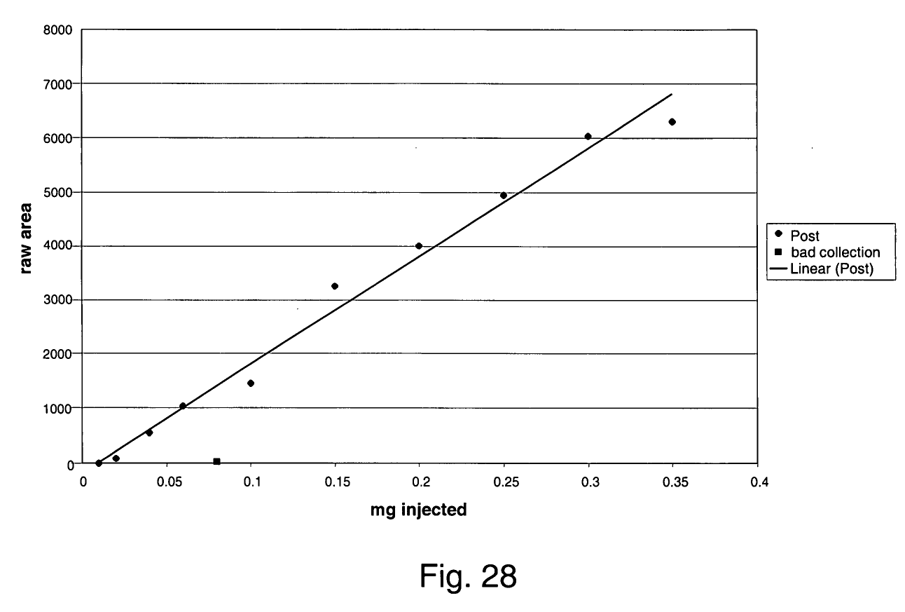

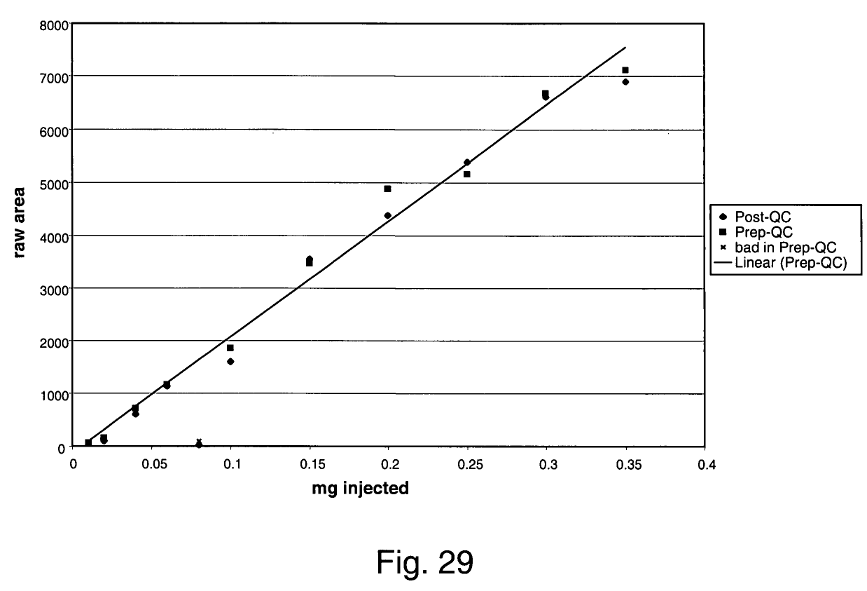

FIG. 27 shows more variation from the line than was observed in FIG. 25. It was postulated that this difference may have been related to small differences in collection efficiency due to small variations in triggering fraction collection. This was confirmed when the collected fractions were evaporated, dissolved, and reanalyzed using the more traditional approach to sample purification and post-purification analysis. The comparison of FIGS. 27 and 28, with the overlay of both sets of data points in FIG. 29, support this.

As the foregoing illustrates, the systems described herein reliably sample amounts that are representative or proportional to the amount of material collected during a fraction collection event. Accordingly, the systems and methods described herein permit the immediate analysis of these proportionally sampled amounts from the flow stream, removing additional evaporation steps and other problems associated with pre-existing purification and analysis methods.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A purification system, comprising:
at least a first chromatographic component configured to separate sample components from one another;
at least one fraction collection component that is structured to collect at least one sample fraction;
at least one representative sample fraction storage component that is structured to at least transiently store at least one representative sample fraction aliquot; and,
at least one splitting mechanism in fluid communication with the first chromatographic component, the fraction collection component, and the representative sample fraction storage component, which splitting mechanism is structured to split sample fractions flowed toward the fraction collection component such that substantially representative aliquots of the sample fractions are flowed to the representative sample fraction storage component.

2. The purification system of claim 1, wherein the first chromatographic component comprises a serial device.

3. The purification system of claim 1, wherein the first chromatographic component comprises at least one chromatography column.

4. The purification system of claim 1, comprising at least one handling component that is configured to convey samples into an inlet of the first chromatographic component.

5. The purification system of claim 1, comprising at least one pump operably connected to at least one system component, which pump is configured to convey fluidic materials in the system.

6. The purification system of claim 1, wherein the fraction collection component comprises at least one holder comprising a base, a coupling mechanism, and a top plate comprising a plurality of apertures, wherein the coupling mechanism couples the base to the top plate in at least one position.

7. The purification system of claim 6, wherein the top plate and the base have disposed between them one or more structures collectively comprising a plurality of external processing regions.

8. The purification system of claim 7, wherein at least one body structure is disposed on the top plate such that the top plate is between the body structure and the structures comprising the external processing regions, the body structure comprising a plurality of first access apertures connected to, and separated from, a plurality of second access apertures by a plurality of inner cavities, the inner cavities comprising a plurality of internal processing regions; wherein the body and the structures are removably sealed such that the internal processing regions are removably sealed to the external processing regions.

9. The purification system of claim 1, wherein the splitting mechanism fluidly communicates with the fraction collection component and the representative sample fraction storage component via at least one conduit.

10. The purification system of claim 9, wherein at least a portion of the conduit comprises the representative sample fraction storage component.

11. The purification system of claim 1, comprising one or more switching valves in fluid communication with at least one system component.

12. The purification system of claim 11, wherein the representative sample fraction storage component comprises at least one fluid loop disposed at least partially in at least one of the switching valves.

13. The purification system of claim 1, comprising at least one controller operably connected to at least one system component, which controller controls operation of the system component.

14. The purification system of claim 13, wherein the controller comprises at least one computer having one or more logic instructions that effect one or more of:
receiving one or more user input parameters selected from the group consisting of: sample input volumes, fluid flow rates, solvent selection, gradient selection, flow path selection, representative sample fraction aliquot volumes, collected sample fraction volumes, delay time between sample component detection and sample fraction aliquot collection and/or storage, at least one fraction collection component volume capacity, and output displays;
tracking samples, sample fractions, and/or representative sample fraction aliquots;
receiving and storing data from the system component;
correlating data received from the system component with one or more sample fractions collected by the fraction collection component;
processing data to produce processed data;
terminating further processing of one or more sample fractions and/or another sample if processed data fails to meet one or more selected criteria;
quantifying one or more sample fraction components;
altering a duration of time between detection of separated sample components and triggering sample fraction collection and representative sample fraction aliquot storage;
altering a delay duration between detection of separated sample components and triggering sample fraction collection;
altering a duration of time between triggering sample fraction collection and triggering representative sample fraction aliquot storage; or,
conveying samples into an inlet of the first chromatographic component.

15. The purification system of claim 1, comprising at least a second chromatographic component in fluid communication with the representative sample fraction storage component, which second chromatographic component is configured to separate components of representative sample fraction aliquots from one another when the representative sample fraction aliquots are conveyed from the representative sample fraction storage component into an inlet of the second chromatographic component.

16. The purification system of claim 15, wherein the second chromatographic component and the representative sample fraction storage component together comprise a parallel device.

17. The purification system of claim 15, wherein the first and/or second chromatographic component comprises a supercritical fluid chromatographic component.

18. The purification system of claim 15, wherein the first and second chromatographic components comprise identical types of chromatographic components.

19. The purification system of claim 15, wherein the first and second chromatographic components comprise different types of chromatographic components.

20. The purification system of claim 15, wherein the second chromatographic component comprises at least one chromatography column.

21. The purification system of claim 15, comprising at least one detection component that communicates with both the first and second chromatographic components.

22. The purification system of claim 21, wherein the detection component comprises one or more of: a mass spectrometer, a UV/Vis detector, an evaporative light-scattering detector, a nuclear magnetic resonance detector, an electrochemical detector, a fluorescence detector, a chemiluminescent nitrogen detector, a refractive index detector, a thermal conductivity detector, a flame ionization detector, a photoionization detector, an electron capture detector, a radiation detector, or a weight scale.

23. A computer program product comprising a computer readable medium having one or more logic instructions that effect collection of at least a first aliquot of at least one sample fraction in at least one fraction collection component of a purification system and at least transient storage of at least a second aliquot of the sample fraction in at least one representative sample fraction storage component of the purification system when the sample fraction is split into at least two aliquots that are substantially representative of one another in the purification system.

24. The computer program product of claim 23, comprising at least one logic instruction that effects separation of two or more components of a sample from one another with at least one chromatographic component of the purification system to produce the sample fraction.

25. The computer program product of claim 23, comprising at least one logic instruction that effects one or more of:
receiving one or more user input parameters selected from the group consisting of: sample input volumes, fluid flow rates, solvent selection, gradient selection, flow path selection, representative sample fraction aliquot volumes, collected sample fraction volumes, delay time between sample component detection and sample fraction aliquot collection and/or storage, at least one fraction collection component volume capacity, and output displays;
tracking samples, sample fractions, and/or representative sample fraction aliquots;
receiving and storing data from at least one component of the purification system;
altering a duration of time between detection of separated sample components and triggering sample fraction collection and representative sample fraction aliquot storage;
altering a delay duration between detection of separated sample components and triggering sample fraction collection;
altering a duration of time between triggering sample fraction collection and triggering representative sample fraction aliquot storage; or,
correlating the data received from the purification system component with one or more sample fractions collected by the fraction collection component.

26. The computer program product of claim 23, wherein the computer readable medium comprises one or more of: a CD-ROM, a floppy disk, a tape, a flash memory device or component, a system memory device or component, a hard drive, or a data signal embodied in a carrier wave.

27. The computer program product of claim 23, comprising at least one logic instruction that effects detection of at least one property of the sample fraction, the first aliquot of the sample fraction, and/or the second aliquot of the sample fraction with at least one detection component of the purification system.

28. The computer program product of claim 27, comprising at least one logic instruction for correlating the detected property of the second aliquot of the sample fraction with the first aliquot of the first sample fraction collected in the fraction collection component to produce correlation data.

29. The computer program product of claim 28, comprising at least one logic instruction that effects storage of the correlation data for the first and second aliquots in an identical data file in at least one data storage medium.

30. The computer program product of claim 28, comprising at least one logic instruction for automatically processing the correlation data to produce processed correlation data.

31. The computer program product of claim 30, comprising at least one logic instruction that effects termination of further processing of the sample fraction and/or another sample if the processed correlation data fails to meet one or more selected criteria.

32. The computer program product of claim 31, comprising at least one logic instruction that effects introduction of another sample into the purification system.

33. The computer program product of claim 23, comprising at least one logic instruction that effects separation of components of the second aliquot of the sample fraction from one another with at least one chromatographic component of the purification system.

34. The computer program product of claim 33, comprising at least one logic instruction that effects detection of the separated components of the second aliquot of the sample fraction with at least one detection component of the purification system.

35. The computer program product of claim 23, comprising at least one logic instruction that effects quantification of at least one component of the sample fraction.

36. The computer program product of claim 35, comprising at least one logic instruction for altering a duration of first aliquot collection to alter a quantity of the component collected in the fraction collection component.

37. A method of storing a sample fraction aliquot, the method comprising:
(a) separating two or more components of the sample from one another to produce at least first and second sample fractions;
(b) splitting at least the first sample fraction into at least two aliquots, which aliquots are substantially representative of one another;
(c) collecting at least a first aliquot of the first sample fraction in at least one fraction collection component; and,
(d) storing at least a second aliquot of the first sample fraction at least transiently in at least one representative sample fraction storage component, thereby storing the sample fraction aliquot.

38. The method of claim 37, comprising optimizing an amount of the first sample fraction that is collected in the fraction collection component.

39. The method of claim 37, comprising performing (a) using at least one chromatographic component.

40. The method of claim 37, comprising quantifying at least one component of the first and/or second sample fractions.

41. The method of claim 37, comprising further processing the first aliquot of the first sample fraction collected in the fraction collection component.

42. The method of claim 37, comprising tracking the sample, the sample fractions, and/or aliquots of the sample fractions.

43. The method of claim 37, comprising performing (c) and (d) substantially simultaneous with one another.

44. The method of claim 37, comprising altering a duration of (c) to alter a quantity of the first sample fraction collected in the fraction collection component.

45. The method of claim 44, wherein (c) comprises detecting the first sample fraction and triggering collection of the first sample fraction in the fraction collection component, and wherein the duration of (c) is altered by altering a delay duration between detection of the first sample fraction and triggering the collection of the first sample fraction.

46. The method of claim 37, wherein the second aliquot of the first sample fraction comprises less than 50% of a total volume of the first sample fraction.

47. The method of claim 46, wherein the second aliquot of the first sample fraction comprises less than 20% of a total volume of the first sample fraction.

48. The method of claim 37, comprising:
(e) detecting at least one property of the first sample fraction, the second sample fraction, the first aliquot of the first sample fraction, and/or the second aliquot of the first sample fraction.

49. The method of claim 48, comprising detecting the property of the first sample fraction, the second sample fraction, the first aliquot of the first sample fraction, and/or the second aliquot of the first sample fraction using at least one identical detection component.

50. The method of claim 48, comprising performing (e) prior to separating components of another sample.

51. The method of claim 48, wherein (e) comprises:
(i) separating components of the second aliquot of the first sample fraction from one another; and,
(ii) detecting the separated components.

52. The method of claim 51, comprising performing (i) using at least one chromatographic component.

53. The method of claim 48, comprising correlating the detected property of the second aliquot of the first sample fraction with the first aliquot of the first sample fraction collected in the fraction collection component to produce correlation data.

54. The method of claim 53, comprising automatically processing the correlation data to produce processed correlation data.

55. The method of claim 54, comprising terminating further processing of at least the first sample fraction and/or another sample if the processed correlation data fails to meet one or more selected criteria.

56. The method of claim 53, comprising storing the correlation data in at least one data storage medium.

57. The method of claim 56, comprising storing the correlation data for the first and second aliquots in an identical data file in the data storage medium.

58. The method of claim 37, comprising:
(e) splitting the second sample fraction into at least two aliquots, which aliquots of the second sample fraction are substantially representative of one another;
(f) collecting at least a first aliquot of the second sample fraction in the fraction collection component; and,
(g) storing at least a second aliquot of the second sample fraction at least transiently in the representative sample fraction storage component.

59. The method of claim 58, comprising substantially simultaneously detecting at least one property of the first aliquots of the first and second sample fractions collected in the fraction collection component.
